SEQUENCE LISTING (24 sequences)

SEQ ID NO: 1 - DNA, 24 bp, Artificial Sequence
  CpG ODN
  misc_feature (1)..(24): Bases are phosphorothioate
tcgtcgttttgtcgttttgtcgtt

SEQ ID NO: 2 - DNA, 20 bp, Artificial Sequence
  CpG ODN
  misc_feature (1)..(20): Bases are phosphorothioate
tccatgacgttcctgacgtt

SEQ ID NO: 3 - DNA, 22 bp, Artificial Sequence
  CpG ODN
tgactgtgaacgttcgagatga

SEQ ID NO: 4 - protein, 479 aa, Artificial Sequence
  GITR binding peptide
EVQLLESGGGEVQPGGSLRLSCAASGSVFSIDAMGWYRQAPGKQRELVAVLSGISSAKYAASAPGRFTISRDNAKNTVYLQMSSLRAEDTAVYYCYADVSTGWGRDAHGYWGQGTLVTVKPGGSGGSEVQLLESGGGEVQPGGSLRLSCAASGSVFSIDAMGWYRQAPGKQRELVAVLSGISSAKYAASAPGRFTISRDNAKNTVYLQMSSLRAEDTAVYYCYADVSTGWGRDAHGYWGQGTLVTVKPGGGGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 119 aa, Artificial Sequence
  GITR binding domain
EVQLLESGGGEVQPGGSLRLSCAASGSVFSIDAMGWYRQAPGKQRELVAVLSGISSAKYAASAPGRFTISRDNAKNTVYLQMSSLRAEDTAVYYCYADVSTGWGRDAHGYWGQGTLVTV

SEQ ID NO: 6 - protein, 10 aa, Artificial Sequence
  CDR
SGSVFSIDAM

SEQ ID NO: 7 - protein, 8 aa, Artificial Sequence
  CDR2
LSGISSAK

SEQ ID NO: 8 - protein, 16 aa, Artificial Sequence
  CDR3
YADVSTGWGRDAHGYW

SEQ ID NO: 9 - protein, 218 aa, Homo sapiens
PAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 215 aa, Artificial Sequence
  Human IgG1 Fc deletion mutant at E233, L234, L235
PAPGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 217 aa, Homo sapiens
PAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 12 - protein, 218 aa, Homo sapiens
PAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 13 - protein, 218 aa, Homo sapiens
PAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 218 aa, Homo sapiens
PAPELLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 14 aa, Artificial Sequence
  IgG hinge region
EPKSSDKTHTCPPC

SEQ ID NO: 16 - protein, 9 aa, Artificial Sequence
  IgG hinge region
DKTHTCPPC

SEQ ID NO: 17 - protein, 11 aa, Artificial Sequence
  IgG hinge region
ESKYGPPCPPC

SEQ ID NO: 18 - protein, 6 aa, Artificial Sequence
  Linker sequence
GGSGGS

SEQ ID NO: 19 - protein, 9 aa, Artificial Sequence
  Linker sequence
GGSGGSGGS

SEQ ID NO: 20 - protein, 12 aa, Artificial Sequence
  Linker sequence
GGSGGSGGSGGS

SEQ ID NO: 21 - protein, 15 aa, Artificial Sequence
  Linker sequence
GGSGGSGGSGGSGGS

SEQ ID NO: 22 - protein, 4 aa, Artificial Sequence
  Linker sequence
GGGG

SEQ ID NO: 23 - protein, 5 aa, Artificial Sequence
  Linker sequence
GGGGG

SEQ ID NO: 24 - protein, 6 aa, Artificial Sequence
  Linker sequence
GGGGGG

(19) United States
(12) Patent Application Publication
(10) Pub. No.: US 2019/0263919 A1
(43) Pub. Date: Aug. 29, 2019

(54) COMBINED ANTI TUMOR THERAPY WITH A GITR AGONIST AND CPG

(71) Applicant: Five Prime Therapeutics, Inc., South San Francisco, CA (US)

(72) Inventors: Susan Foy, Mountain View, CA (US); Thomas Brennan, Cupertino, CA (US); Danielle Kellar, San Francisco, CA (US)

(73) Assignee: Five Prime Therapeutics, Inc., South San Francisco, CA (US)

(21) Appl. No.: 16/310,725

(22) PCT Filed: Jun. 30, 2017
(86) PCT No.: PCT/US2017/040259

§ 371 Date: Dec. 17, 2018

Related U.S. Application Data
(60) Provisional application No. 62/357,750, filed on Jul. 1, 2016.

(51) Int. Cl. C07K 16/28; A61K 35/17; A61P 35/00; A61K 31/7125; A61K 9/00; A61K 38/17

(52) U.S. Cl. CPC C07K 16/2878; A61K 35/17; A61K 38/1774; A61K 31/7125; A61K 9/0019; A61P 35/00

(57) ABSTRACT

The present disclosure relates to methods of using glucocorticoid-induced TNFR-related protein (GITR) agonists as anti-tumor agents, for example in combination with CpG oligodeoxynucleotides. A specific antibody, characterized by its CDRs is described as agonist.

### CROSS REFERENCE TO RELATED APPLICATIONS

This application claims the benefit of U.S. Provisional Application No. 62/357,750, filed Jul. 1, 2016, which is herein incorporated by reference in its entirety.

### FIELD

This application relates to the use of glucocorticoid-induced TNFR-related protein (GITR) agonists as anti-tumor agents, for example in combination with CpG oligodeoxynucleotides (ODNs). Also provided are kits and compositions that can be used with such methods.

### BACKGROUND

Cancer is a group of diseases involving abnormal cell growth with the potential to invade or spread to other parts of the body. In 2012 about 14.1 million new cases of cancer occurred globally (not including skin cancer other than melanoma). It caused about 8.2 million deaths or 14.6% of human deaths. The most common types of cancer in males are lung cancer, prostate cancer, colorectal cancer and stomach cancer. In females, the most common types are breast cancer, colorectal cancer, lung cancer and cervical cancer. There is a continued need for effective treatments for cancers, and their metastases.

### SUMMARY

Provided herein are methods for treating a tumor in a mammal, such as a human, veterinary animal or laboratory animal. The methods include administration of an effective amount of at least one glucocorticoid-induced TNFR-related protein (GITR) agonist (such as intratumoral or systemic administration) and intratumoral administration of an effective amount of at least one CpG ODN, thereby treating the tumor in the mammal. In some examples, the at least one GITR agonist and the at least one CpG ODN are administered simultaneously or contemporaneously. In some examples, the at least one GITR agonist and the at least one CpG ODN are administered on at least two different occasions at least twice, such as 2, 3, 4, 5, 6, 7, 8, 9 or 10 times. In some examples, both the GITR agonist and the CpG ODN are administered in multiple doses, at least 1 day, at least 2 days, at least 5 days, at least 7 days at least 14 days, or at least 30 days apart. In some examples, the method further includes administering an effective amount of one or more additional therapeutic agents, such as one or more chemotherapeutic agents, one or more biologic agents, one or more anti-angiogenesis agents, one or more growth inhibitory agents, one or more anti-neoplastic compositions, surgery, or combinations thereof.

Exemplary GITR agonists include a GITR antibody or GITR antibody fragment, (such as a chimeric antibody, humanized antibody, human antibody, bispecific antibody, single chain antibody, Fv, a single-chain Fv (scFv), a Fab, a Fab′, or a (Fab′)₂), a large molecule, a small molecule, or a GITR encoding nucleic acid molecule. In a specific example, the at least one GITR agonist is a GITR antibody fragment selected from an Fv, a single-chain Fv (scFv), a Fab, a Fab′, and a (Fab′)₂. In a specific example, the at least one GITR agonist is a GITR antibody is selected from a) an antibody comprising a GITR binding domain (GITR-BD) comprising a CDR1 comprising the sequence of SEQ ID NO: 6, a CDR2 comprising the sequence of SEQ ID NO: 7, and a CDR3 comprising the sequence of SEQ ID NO: 8; b) an antibody comprising a GITR-BD comprising the sequence of SEQ ID NO: 5; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 6, a CDR2 comprising the sequence of SEQ ID NO: 7, and a CDR3 comprising the sequence of SEQ ID NO: 8, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:5, (ii) the Linker is a polypeptide, (iii) the Hinge is a polypeptide derived from an immunoglobulin hinge region, and (iv) the Fc is an immunoglobulin Fc polypeptide; and e) a tetravalent molecule comprising two copies of a polypeptide comprising the sequence of SEQ ID NO: 4.

Exemplary CpG ODNs include class B ODNs, such as the human CpG sequence shown in SEQ ID NO: 1 (5′-tcgtcgttttgtcgttttgtcgtt-3′ with bases that are phosphorothioate) and the murine CpG sequence shown in SEQ ID NO: 2 (5′-tccatgacgttcctgacgtt-3′ with bases that are phosphorothioate). In one example, the CpG ODNs has the sequence 5′-TGACTGTGAACGTTCGAGATGA-3′ SEQ ID NO: 3 (ISS 1018 from Dynavax). Other examples of are provided in Rachmilewitz et al. (Inflammatory Bowel Diseases 12(5):339-45, 2006). One skilled in the art will appreciate that other TLR9 agonists can be used as an alternative (or in addition) to a CpG ODN.

In some examples, the effective amount of the at least one GITR agonist is at least 0.01 mg/kg, at least 0.03 mg/kg, at least 0.25 mg/kg, at least 0.3 mg/kg, or at least 1 mg/kg. In some examples, the effective amount of the at least one GITR agonist is no more than 1 mg/kg, no more than 0.3 mg/kg, no more than 0.25 mg/kg, no more than 0.03 mg/kg, no more than 0.01 mg/kg, or no more than 0.01 mg/kg, for example 0.1 mg/kg to 1 mg/kg. In some examples, the effective amount of the at least one CpG ODN is at least 0.05 mg, at least 0.3 mg, at least 1 mg, at least 3 mg, at least 6 mg, at least 18 mg, or at least 20 mg. In some examples, the effective amount of the at least one CpG ODN is no more than 20 mg, no more than 10 mg, no more than 1 mg, no more than 0.3 mg, or no more than 0.05 mg, such as 005 mg to 20 mg. In some examples, the effective amount of the at least one GITR agonist is from 0.01 to 0.1 mg/kg, and the effective amount of the at least one CpG ODN is from 0.5 to 1.0 mg/kg.

Exemplary tumors that can be treated with the disclosed methods include, but are not limited to, a lymphoma, melanoma, sarcoma or adenocarcinoma. In some embodiments, the tumor is a melanoma. In some examples, the tumor is a cancer of the breast, liver, spleen, kidney, colon, prostate, lung, central nervous system, head and neck, stomach, pancreas, ovary, cervix, testis, bladder or gallbladder. In some embodiments, the tumor is selected from breast and colon cancer. Other examples are provided herein. In some examples, the disclosed methods decrease the size or volume of the injected tumor by at least 30%, at least 40%, at least 50%, at least 60%, at least 70%, at least 80%, at least 90%, or at least 95%, for example as compared to no administration of the GITR agonist and CpG ODN. In some examples, the disclosed methods decrease the size or volume of a non-injected metastatic tumor by at least 30%, at least 40%, at least 50%, at least 60%, at least 70%, at least 80%, at least 90%, or at least 95%, for example as compared to no administration of the GITR agonist and CpG ODN. In some examples, the disclosed methods decrease the number of non-injected metastatic tumors by at least 30%, at least 40%, at least 50%, at least 60%, at least 70%, at least 80%, at least 90%, or at least 95%, for example as compared to no administration of the GITR agonist and CpG ODN.

The foregoing and other objects and features of the disclosure will become more apparent from the following detailed description, which proceeds with reference to the accompanying figures.

### BRIEF DESCRIPTION OF THE DRAWINGS

FIG. 1 provides an overview of the method used in Example 1.

FIGS. 2A-2F are graphs showing the results of direct tumor injection of (A, B) CpG, (B, C) anti-GITR or (D, F) both CpG and anti-GITR on tumor regression of the injected tumor or a distant, non-injected tumor. (A) CpG alone caused regression of the injected tumor, but (B) not the distant non-injected tumor. (C) Anti-GITR caused regression of the injected and (D) distant tumors in some mice. (E) The combination of CpG and anti-GITR caused regression of the injected and (F) distant tumors.

FIGS. 3A-3F are graphs showing the results of direct tumor injection of CpG, or i.p. injection of anti-GITR or both direct tumor injection of CpG and i.p. injection anti-GITR on tumor regression of the injected tumor or a distant, non-injected tumor. (A) CpG alone caused regression of the injected tumor, but (B) not the distant non-injected tumor. (C, D) Anti-GITR administered i.p. caused regression of the tumors in some mice. (E) The combination of CpG administered intratumorally and anti-GITR administered i.p. (e.g., systemic anti-GITR) caused regression of the injected tumor and (F) distant tumors in some, but not all mice.

FIGS. 4A-4D provide schematic representations of certain multivalent anti-GITR antibody architectures.

### SEQUENCE LISTING

The nucleic acid sequences are shown using standard letter abbreviations for nucleotide bases and three letter abbreviations for amino acids, as defined in 37 C.F.R. 1.822. Only one strand of each nucleic acid sequence is shown, but the complementary strand is understood as included by any reference to the displayed strand.

SEQ ID NOs: 1-3 show exemplary CpG ODN sequences.

SEQ ID NO: 4 is an exemplary GITR binding polypeptide.

SEQ ID NO: 5 is an exemplary GITR binding domain.

SEQ ID NO: 6 is an exemplary CDR1 polypeptide.

SEQ ID NO: 7 is an exemplary CDR2 polypeptide.

SEQ ID NO: 8 is an exemplary CDR4 polypeptide.

SEQ ID NOs: 9 to 14 show exemplary protein Fc sequences.

SEQ ID NOs: 15 to 17 show exemplary protein hinge sequences.

SEQ ID NOs: 18 to 24 show exemplary protein linker sequences.

### DETAILED DESCRIPTION

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. Dates of GenBank® Accession Nos. referred to herein are the sequences available at least as early as Jul. 1, 2016. All references (including journal articles, patents, and patent applications) and GenBank® Accession numbers cited or referred to herein are incorporated by reference.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Administration: To provide or give a subject an agent, such as one or more agents that enhance or increase GITR activity (e.g., an agonist specific for GITR nucleic acid molecules or proteins or downstream products thereof), and/or one or more CpG ODNs (e.g., a TLR9 agonist), by any effective route. Exemplary routes of administration include, but are not limited to, oral, injection (such as subcutaneous, intramuscular, intradermal, intraperitoneal, intravenous, peritumoral, and intratumoral), sublingual, rectal, transdermal, intranasal, vaginal and inhalation routes. In one example, the route of administration is intratumoral.

Agent: Any substance or any combination of substances that is useful for achieving an end or result; for example, a substance or combination of substances useful for treating a tumor, such as cancer. Agents include, and are not limited to, proteins, nucleic acid molecules, compounds, small molecules, large molecules, organic compounds, inorganic compounds, or other molecules of interest. In some embodiments, the agent is a polypeptide agent (such as an antibody), a nucleic acid molecule (such as a nucleic acid molecule encoding GITR or a CpG ODN) or a pharmaceutical compound. The skilled artisan will understand that particular agents may be useful to achieve more than one result.

Agonist: Any molecule that enhances or increases a biological activity of a protein, such as GITR or TLR9, or that enhances or increases the transcription or translation of a nucleic acid encoding the protein. Exemplary agonist molecules include, but are not limited to, agonist antibodies, agonist peptides, oligopeptides, organic molecules (including large and small molecules), and nucleic acid molecules (e.g., GITR-encoding nucleic acid molecules or a CpG ODN). The term “GITR agonist” refers to a molecule or combination of molecules that directly or indirectly increase or enhance GITR expression and/or activity, for example by agonizing effector T cells, reducing suppressive activity and lineage stability of T regulatory cells, depleting T regulatory cells, or combinations thereof. The term “TLR9 agonist” refers to a molecule or combination of molecules that directly or indirectly increase or stimulate TLR9 expression and/or activity, for example by inducing B cell proliferation and differentiation, and or plasmacytoid (pDC) activation and IFN-alpha secretion.

Antibody: A polypeptide including at least a light chain or heavy chain immunoglobulin variable region which specifically recognizes and binds an epitope of an antigen, such as GITR, or a fragment thereof. Antibodies are composed of a heavy and a light chain, each of which has a variable region, termed the variable heavy (V_{H}) region and the variable light (V_{L}) region. Together, the V_{H} region and the V_{L} region are responsible for binding the antigen recognized by the antibody. Antibodies of the present disclosure include those that are specific for GITR and in some examples also enhance or increase the biological activity of the protein (such as agonizing effector T cells, reducing suppressive activity and lineage stability of T regulatory cells, depleting T regulatory cells, or both).

The term antibody includes intact immunoglobulins, as well the variants and portions thereof, such as Fab′ fragments, F(ab)′₂ fragments, single chain Fv proteins (“scFv”), Fv, Fab, and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (for example, humanized murine antibodies), single-chain antibodies (e.g., camelid antibodies), and heteroconjugate antibodies (such as, bispecific antibodies). See also, Pierce Catalog and Handbook, 1994-1995 (Pierce Chemical Co., Rockford, Ill.); Kuby, J., Immunology, 3^{rd} Ed., W.H. Freeman & Co., New York, 1997.

Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). In combination, the heavy and the light chain variable regions specifically bind the antigen. Light and heavy chain variable regions contain a “framework” region interrupted by three hypervariable regions, also called “complementarity-determining regions” or “CDRs”. The extent of the framework region and CDRs have been defined (see, Kabat et al., Sequences of Proteins of Immunological Interest, U.S. Department of Health and Human Services, 1991). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. An antibody that binds a target protein will have a specific V_{H} region and the V_{L} region sequence, and thus specific CDR sequences. Antibodies with different specificities (such as different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

A “polyclonal antibody” is an antibody that is derived from different B-cell lines. Polyclonal antibodies are a mixture of immunoglobulin molecules secreted against a specific antigen, each recognizing a different epitope. These antibodies are produced by methods known to those of skill in the art, for instance, by injection of an antigen into a suitable mammal (such as a mouse, rabbit or goat) that induces the B-lymphocytes to produce IgG immunoglobulins specific for the antigen, which are then purified from the mammal's serum.

A “chimeric antibody” has framework residues from one species (such as mouse, rat, cynomolgus monkey, etc.) and CDRs from another species (such as human, cynomolgus monkey, chicken, etc.). In some examples, a chimeric antibody includes at least one mouse variable region and at least one human constant region. In some examples, a chimeric antibody includes at least one cynomolgus variable region and at least one human constant region. In some embodiments, all of the variable regions of a chimeric antibody are from a first species and all of the constant regions of the chimeric antibody are from a second species.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” In one example, all the CDRs are from the donor immunoglobulin in a humanized immunoglobulin. Constant regions need not be present, but if they are, they are substantially identical to human immunoglobulin constant regions, e.g., at least about 85-90%, such as about 95% or more identical. Hence, all parts of a humanized immunoglobulin, except possibly the CDRs, are substantially identical to corresponding parts of natural human immunoglobulin sequences. Humanized immunoglobulins can be constructed by means of genetic engineering (see for example, U.S. Pat. No. 5,585,089). In some embodiments, a humanized antibody includes at least one human constant region or fragment thereof. In some embodiments, a humanized antibody is an Fab, an scFv, a (Fab′)₂, etc.

A “CDR-grafted antibody” refers to a humanized antibody in which one or more complementarity determining regions (CDRs) of a first (non-human) species have been grafted onto the framework regions (FRs) of a second (human) species.

Binding: An association between two substances or molecules, such as the hybridization of one nucleic acid molecule to another (or itself), the association of an antibody or small organic molecule with a protein, or the association of a protein with another protein or nucleic acid molecule. Binding can be detected by any procedure known to one skilled in the art, including, but not limited to: Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorptionlionization time-of-flight mass spectrometry, microcytometry, microarray, microscopy, fluorescence activated cell sorting (FACS), and flow cytometry.

One molecule is said to “specifically bind” to another molecule when a particular agent (a “specific binding agent”) can specifically react with a particular target, but not to unrelated molecules, for example to specifically immunoreact with a target, to specifically hybridize to a target, or to specifically bind to a target. For example, a GITR specific binding agent binds substantially only to the GITR protein in vitro or in vivo. The binding is a non-random binding reaction, for example between a specific binding agent (such as an antibody or functional fragment thereof, protein, or nucleic acid molecule) and a target (such as a cell, protein, DNA or RNA). Binding specificity can be determined from the reference point of the ability of the specific binding agent to differentially bind the target and an unrelated molecule, and therefore distinguish between two different molecules. For example, an oligonucleotide molecule binds or stably binds to a target nucleic acid molecule if a sufficient amount of the oligonucleotide molecule forms base pairs or is hybridized to its target nucleic acid molecule, to permit detection of that binding.

In some examples, a molecule (such as an antibody) specifically binds to a target (such as a protein) with a binding constant that is at least 10³ M⁻¹ greater, 10⁴M⁻¹ greater or 10⁵ M⁻¹ greater than a binding constant for other molecules in a sample or subject. In particular examples, two compounds are said to specifically bind when the binding constant for complex formation between the components is at least 10⁴ L/mol, for example, at least 10⁶ L/mol, at least 10⁸ L/mol, or at least 10¹⁰ L/mol. The binding constant for two components can be determined using methods that are well known in the art.

In particular examples, two compounds are said to specifically bind when the binding affinity of at least about 0.1×10⁻⁸ M, at least about 0.3×10⁻⁸ M, at least about 0.5×10⁻⁸ M, at least about 0.75×10⁻⁸ M, at least about 1.0×10⁻⁸ M, at least about 1.3×10⁻⁸ M at least about 1.5×10⁻⁸ M, at least about 2.0×10⁻⁸ M, at least about 2.5×10⁻⁸, at least about 3.0×10⁻⁸, at least about 3.5×10⁻⁸, at least about 4.0×10⁻⁸, at least about 4.5×10⁻⁸, or at least about 5.0×10⁻⁸ M.

In certain embodiments, a specific binding agent that binds to target has a dissociation constant (Kd) of ≤104 nM, ≤100 nM, ≤10 nM, ≤1 nM, ≤0.1 nM, ≤0.01 nM, or ≤0.001 nM (e.g., 10⁻⁸M or less, e.g., from 10⁻⁸M to 10⁻¹³M, e.g., from 10⁻⁹M to 10⁻¹³ M). In one embodiment, Kd is measured by a radiolabeled antigen binding assay (RIA) performed with the Fab version of an antibody of interest and its antigen (see, e.g., Chen et al., J. Mol. Biol. 293:865-881, 1999). In another example, Kd is measured using surface plasmon resonance assays using a BIACORES-2000 or a BIACORES-3000 (BIAcore, Inc., Piscataway, N.J.) at 25° C. with immobilized antigen CMS chips at about 10 response units (RU).

Cancer: A malignant tumor characterized by abnormal or uncontrolled cell growth. Other features often associated with cancer include metastasis, interference with the normal functioning of neighboring cells, release of cytokines or other secretory products at abnormal levels and suppression or aggravation of inflammatory or immunological response, invasion of surrounding or distant tissues or organs, such as lymph nodes, etc. “Metastatic disease” refers to cancer cells that have left the original tumor site and migrate to other parts of the body for example via the bloodstream or lymph system.

Contacting: Placement in direct physical association; includes both in solid and liquid form, which can take place either in vivo or in vitro. Contacting includes contact between one molecule and another molecule, for example between a protein and an antibody. Contacting can also include contacting a cell or tissue, for example by placing a test agent in direct physical association with a cell or tissue (such as a tumor sample) or by administration of an agent to a subject.

Control: A reference standard. In some embodiments, the control is a result expected in the absence of a therapeutic agent (such as no substantial immune effect of GITR activity, TLR9 activity, or both, for example against a tumor cell). In some embodiments, the control is a result expected in the presence of an agent that increases immune mediated GITR activity and/or expression, for example against a tumor cell. In some embodiments, the control is a result expected in the presence of an agent that increases immune mediated TLR9 activity and/or expression, for example against a tumor cell. In some embodiments, the control is a result expected in the absence of an agent that increases immune mediated GITR activity and/or expression, for example against a tumor cell. In some embodiments, the control is a result expected in the absence of an agent that increases immune mediated TLR9 activity and/or expression, for example against a tumor cell. In still other embodiments, the control is a historical control or standard reference value or range of values (such as a previously tested control sample, such as a group of patients with known prognosis or outcome, or group of samples that represent baseline or normal values). In some examples, a control sample or value refers to a sample, cell or tissue obtained from a source known, or believed, not to be afflicted with the disease or condition for which a method or composition of the provided herein is being used to identify. In one embodiment, a reference sample, reference cell or reference tissue is obtained from a healthy part of the body of the same subject or patient in whom a disease or condition is being identified using a composition or method provided herein. In one embodiment, a reference sample, reference cell or reference tissue is obtained from a healthy part of the body of at least one individual who is not the subject or patient in whom a disease or condition is being identified using a composition or method provided herein. In some embodiments, a reference sample, reference cell or reference tissue was previously obtained from a patient prior to developing a disease or condition or at an earlier stage of the disease or condition.

A difference between a test sample and a control can be an increase or conversely a decrease. The difference can be a qualitative difference or a quantitative difference, for example a statistically significant difference. Suitable statistical analyses are well known in the art, and include, but are not limited to, Student's T test and ANOVA assays. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 95%, at least about 96%, at least about 97%, at least about 98%, at least about 99%, at least about 100%, at least about 150%, at least about 200%, at least about 250%, at least about 300%, at least about 350%, at least about 400%, at least about 500%, or greater than 500%.

CpG oligodeoxynucleotide (ODN): A short synthetic single-stranded DNA molecule containing unmethylated CpG dinucleotides in particular sequence contexts (CpG motifs). CpG ODNs are recognized by Toll-like receptor 9 (TLR9) leading to strong immunostimulatory effects, and thus are TLR9 agonists. CpG ODNs possess a partially or completely phosphorothioated (PS) backbone, as opposed to the natural phosphodiester (PO) backbone found in genomic bacterial DNA. Three major classes of stimulatory CpG ODNs have been identified based on structural characteristics and activity on human peripheral blood mononuclear cells (PBMCs), in particular B cells and plasmacytoid dendritic cells (pDCs). These three classes are Class A (Type D), Class B (Type K) and Class C.

Class B (type K) CpG ODNs (CpG-B ODNs) are 18-28mer linear oligodeoxynucelotides. They contain a fully phosphorothioated backbone with one or more 6mer CpG motifs. In one example, the optimal motif is GTCGTT in human and GACGTT in mouse. CpG-B ODNs stimulate strong B cell and plasmacytoid dendritc cell (pDC) activation but are weak inducers of IFN-α secretion. They display anti-tumor activity and are potent Th1 adjuvants. Exemplary Class B CpG ODNs include human CpG ODN 2006 (5′-tcgtcgttttgtcgttttgtcgtt-3′, with bases that are phosphorothioate SEQ ID NO: 1), murine ODN 1826 (5′-tccatgacgttcctgacgtt-3′ with bases that are phosphorothioate SEQ ID NO: 2), and 5′-TGACTGTGAACGTTCGAGATGA-3′ (SEQ ID NO: 3).

In some examples, a Class A or Class C CpG ODN is used in the methods provided herein.

Decrease: To reduce the quality, amount, or strength of something by a statistically significant amount, relative to a control value. In one example, a therapy decreases the size or volume of a tumor, the number of tumors or metastases, or combinations thereof, for example as compared to the response in the absence of the therapy. In some examples a decrease is a decrease of at least 20%, at least 25%, at least 50%, at least 75%, at least 80%, at least 90%, at least 95%, at least 98%, at least 99%, or at least 100%, as compared to a control value.

Expression: The process by which the coded information of a nucleic acid molecule, such as a GITR nucleic acid molecule (e.g., GITR gene) is converted into an operational, non-operational, or structural part of a cell, such as the synthesis of a protein (e.g., GITR protein). Expression of a gene can be regulated anywhere in the pathway from DNA to RNA to protein. Regulation can include controls on transcription, translation, RNA transport and processing, degradation of intermediary molecules such as mRNA, or through activation, inactivation, compartmentalization or degradation of specific protein molecules after they are produced.

The expression of a nucleic acid molecule or protein (such as GITR) can be altered relative to a normal clinical pathology (such as in a normal non-cancerous cell), or relative to an abnormal clinical pathology (such as in a tumor cell). Alterations in gene expression, such as differential expression, include but are not limited to: (1) overexpression (e.g., upregulation); (2) underexpression (e.g., downregulation); or (3) suppression of expression. Alternations in the expression of a nucleic acid molecule can be associated with, and in fact cause, a change in expression of the corresponding protein.

Protein expression can also be altered in some manner to be different from the expression of the protein in a normal situation. This includes but is not necessarily limited to: (1) a mutation in the protein such that one or more of the amino acid residues is different; (2) a short deletion or addition of one or a few (such as no more than 10-20) amino acid residues to the sequence of the protein; (3) a longer deletion or addition of amino acid residues (such as at least 20 residues), such that an entire protein domain or sub-domain is removed or added; (4) expression of an increased amount of the protein compared to a control or standard amount (e.g., upregulation); (5) expression of a decreased amount of the protein compared to a control or standard amount (e.g., downregulation); (6) alteration of the subcellular localization or targeting of the protein; (7) alteration of the temporally regulated expression of the protein (such that the protein is expressed when it normally would not be, or alternatively is not expressed when it normally would be); (8) alteration in stability of a protein through increased longevity in the time that the protein remains localized in a cell; and (9) alteration of the localized (such as organ or tissue specific or subcellular localization) expression of the protein (such that the protein is not expressed where it would normally be expressed or is expressed where it normally would not be expressed), each compared to a control or standard.

Controls or standards for comparison to a sample, for the determination of differential expression, include samples believed to be normal (in that they are not altered for the desired characteristic, for example a cell from a healthy subject), samples believed to be abnormal (in that they are altered for the desired characteristic, for example a tumor cell), as well as laboratory values, even though possibly arbitrarily set, keeping in mind that such values can vary from laboratory to laboratory. Laboratory standards and values may be set based on a known or determined population value and can be supplied in the format of a graph or table that permits comparison of measured, experimentally determined values.

Glucocorticoid-induced TNFR-related protein (GITR): Also known as tumor necrosis factor receptor superfamily member 18 (TNFRSF18) and activation-inducible TNFR family receptor (AITR). Includes GITR nucleic acid molecules and proteins (e.g., OMIM 603905). GITR is a cell surface receptor transmembrane protein constitutively expressed on Treg cells, and is upregulated when T cells are activated. GITR has been shown to be involved in inhibiting the suppressive activity of T-regulatory cells and extending the survival of T-effector cells. Human GITR has three isoforms and mouse GITR has two isoforms. GITR is activated by GITR ligand (GITRL).

GITR sequences are publically available, for example from the GenBank® sequence database (e.g., Accession Nos. NP_683700.1 (isoform 3 precursor, mature peptide is aa 26-234), NP_683699.1 (isoform 2 precursor, mature peptide is aa 26-255), NP_004186.1 (isoform 1 precursor, mature peptide is aa 26-241), NP_033426.1 (isoform 1 precursor, mature peptide is aa 20-228) and NP_068820.1 (isoform 2 precursor, mature peptide is aa 22-132) provide exemplary GITR protein sequences, while Accession Nos. NM_148902.1, NM_148901.1, NM_004195.2, NM_009400.2, and NM_021985.2 provide exemplary GITR nucleic acid sequences). One of ordinary skill in the art can identify additional GITR or GITRL nucleic acid and protein sequences, including GITR antibodies and GITRL variants that retain the ability to bind GITR, agonize effector T cells, reduce suppressive activity and lineage stability of T regulatory cells, deplete T regulatory cells, or combinations thereof.

Host cell: A cell that may be or has been a recipient of a vector or isolated polynucleotide. Host cells may be prokaryotic cells or eukaryotic cells. Exemplary eukaryotic cells include mammalian cells, such as tumor cells.

Inhibit or reduce: A decrease or cessation of any phenotypic characteristic or to the decrease or cessation in the incidence, degree, or likelihood of that characteristic. In some embodiments, by “reduce” or “inhibit” is meant the ability to cause a decrease of at least 10%, or at least 20%, at least 30%, at least 40%, at least 50%, at least 75%, at least 80%, at least 90%, at least 95%, at least 98% or at least 99%.

Isolated: An “isolated” biological component (such as a GITR protein, antibody, or nucleic acid molecule, or a CpG ODN) has been substantially separated, produced apart from, or purified away from other biological components in which the component occurs, such as other chromosomal and extrachromosomal DNA and RNA, and proteins. Nucleic acids molecules and proteins which have been “isolated” thus include nucleic acids and proteins purified by standard purification methods. The term also embraces nucleic acid molecules and proteins prepared by recombinant expression in a host cell as well as chemically synthesized nucleic acids. A purified or isolated cell, antibody, protein, or nucleic acid molecule can be at least 70%, at least 80%, at least 90%, at least 95%, at least 96%, at least 97%, at least 98%, or at least 99% pure.

Mammal: This term includes both human and non-human mammals. Similarly, the term “subject” includes both human and veterinary subjects (such as cats, dogs, horses, cows, and pigs) and rodents (such as mice and rats).

Mean and Standard Deviation: The arithmetic mean is the “standard” average, often simply called the “mean”.

$$\overline{x} - \frac{1}{n} \cdot {\sum }_{i = 1}^{n} {x}_{i}$$

The mean is the arithmetic average of a set of values.

The standard deviation (represented by the symbol sigma, σ) shows how much variation or “dispersion” exists from the mean. The standard deviation of a random variable, statistical population, data set, or probability distribution is the square root of its variance. The standard deviation is commonly used to measure confidence in statistical conclusions. Generally, twice the standard deviation is about the radius of a 95 percent confidence interval. Effects that fall far outside the range of standard deviation are generally considered statistically significant. One of skill in the art can readily calculate the mean and the standard deviation from a population of values.

Nucleic acid molecule: A polymer of nucleotides, which may contain natural and/or non-natural nucleotides. Examples include, but are not limited to, DNA, RNA, and PNA.

Pharmaceutically acceptable carriers: The pharmaceutically acceptable carriers useful in this invention are conventional. Remington's Pharmaceutical Sciences, by E. W. Martin, Mack Publishing Co., Easton, Pa., 15th Edition (1975), describes compositions and formulations suitable for pharmaceutical delivery of the agents provided herein such as one or more agents that enhance or increase GITR activity (e.g., a GITR agonist) and/or a CpG ODN (e.g., a TLR9 agonist).

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Purified: The term purified does not require absolute purity; rather, it is intended as a relative term. Thus, for example, a purified peptide or antibody preparation is one in which the peptide or protein is more enriched than the peptide or protein is in its natural environment within a cell. In one embodiment, a preparation is purified such that the protein or antibody represents at least 50%, 60%, 70%, 80%, 90%, 95%, 96%, 97%, 98%, or 99% of the total antibody or protein content of the preparation.

Sample: Material that is obtained or derived from a subject of interest that contains a cellular and/or other molecular entity that is to be characterized and/or identified, for example based on physical, biochemical, chemical and/or physiological characteristics. In some examples, the sample is a tissue or cell sample, such as a fresh, frozen and/or preserved organ or tissue sample or biopsy or aspirate (such as a tumor biopsy or aspirate); blood or any blood constituents; bodily fluids such as sputum, cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like. In one example, the sample is a tumor or cancer sample.

Sequence identity of amino acid or nucleic acid sequences: The similarity between amino acid (or nucleic acid) sequences is expressed in terms of the similarity between the sequences, otherwise referred to as sequence identity. Sequence identity is frequently measured in terms of percentage identity (or similarity or homology); the higher the percentage, the more similar the two sequences are.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Variants of a native GITR protein are typically characterized by possession of at least about 80%, at least 90%, at least 95%, at least 96%, at least 97%, at least 98% or at least 99% sequence identity counted over the full length alignment with the amino acid sequence using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or at least 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Thus, a variant GITR protein can have at least 80%, at least 85%, at least 90%, at least 91%, at least 92%, at least 95%, at least 96%, at least 97%, at least 98% or at least 99% sequence identity to any of the protein sequences shown in GenBank® Accession Nos. NP_683700.1 (isoform 3 precursor, mature peptide is aa 26-234), NP_683699.1 (isoform 2 precursor, mature peptide is aa 26-255), NP_004186.1 (isoform 1 precursor, mature peptide is aa 26-241), NP_033426.1 (isoform 1 precursor, mature peptide is aa 20-228) and NP_068820.1 (isoform 2 precursor, mature peptide is aa 22-132), wherein the variant has the ability to agonize effector T cells, reduce suppressive activity and lineage stability of T regulatory cells, deplete T regulatory cells, or combinations thereof.

Nucleic acids that “selectively hybridize” or “selectively bind” do so under moderately or highly stringent conditions that excludes non-related nucleotide sequences. In nucleic acid hybridization reactions, the conditions used to achieve a particular level of stringency will vary, depending on the nature of the nucleic acids being hybridized. For example, the length, degree of complementarity, nucleotide sequence composition (for example, GC v. AT content), and nucleic acid type (for example, RNA versus DNA) of the hybridizing regions of the nucleic acids can be considered in selecting hybridization conditions. An additional consideration is whether one of the nucleic acids is immobilized, for example, on a filter.

A specific example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (e.g., Molecular Cloning: A Laboratory Manual, 2nd ed., vol. 1-3, ed. Sambrook et al., Cold Spring Harbor Laboratory Press, Cold Spring Harbor, N.Y., 1989). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Thus, exemplary GITR nucleic acid sequences in some examples have at least 70%, at least 80%, at least 85%, at least 90%, at least 92%, at least 95%, at least 98%, or at least 99% sequence identity to any of the nucleic acid sequences shown in GenBank® Accession Nos. NM_148902.1, NM_148901.1, NM_004195.2, NM_009400.2, and NM_021985.2, including GITR variants that retain the ability to bind GITRL, agonize effector T cells, reduce suppressive activity and lineage stability of T regulatory cells, deplete T regulatory cells, or combinations thereof.

Small molecule: A molecule, typically with a molecular weight less than about 1000 Daltons, or in some embodiments, less than about 500 Daltons, wherein the molecule is capable of modulating (e.g., increasing), to some measurable extent, an activity of a target molecule, such as GITR.

Subject: Any mammal, such as humans, non-human primates, pigs, sheep, horses, cows, dogs, cats, rodents and the like which is to be the recipient of the particular treatment, such as treatment with one or more agents that enhance or increase GITR expression and/or activity (e.g., an agonist specific for a GITR) and a CpG ODN. In two non-limiting examples, a subject is a human subject or a murine subject. In some examples, the subject has a tumor, such as cancer. In some examples, the subject has a metastasis.

Therapeutically effective amount: An amount of one or more agents that enhance or increase GITR activity (e.g., a GITR agonist), that alone, or together with an additional therapeutic agent(s) (such as a CpG ODN or other TLR9 agonist), is sufficient to treat a disease or reduce and/or ameliorate the symptoms and/or underlying causes of any of a disorder or disease. In one embodiment, an “effective amount” is sufficient to reduce or eliminate a symptom of a disease, such as a cancer, for example by reducing tumor size, tumor volume, metastasis size, metastasis volume, tumor burden, number of metastases, or combinations thereof.

A therapeutically effective amount of one or more agents that enhance or increase GITR expression and/or activity (e.g., a GITR agonist) and one or more CpG ODNs, can be administered in a single dose, or in several doses, for example daily, during a course of treatment. However, the therapeutically effective amount can depend on the subject being treated, the severity and type of the condition being treated, the manner of administration and the type of therapeutic agent being administered.

Tissue: A plurality of functionally related cells. A tissue can be a suspension, a semi-solid, or solid. Tissue includes cells collected from a subject, such as a tumor.

Treating a disease: “Treatment” refers to a therapeutic intervention that ameliorates a sign or symptom of a disease or pathological condition after it has begun to develop, such a sign or symptom of a cancer. Includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process. Treatment can also induce remission or cure of a condition, such as a cancer. Treatment does not require a total absence of disease. For example, a decrease of at least 20% or at least 50% can be sufficient. The beneficial effect can be evidenced, for example, by a delayed onset of clinical symptoms of the disease in a subject, a reduction in severity of some or all clinical symptoms of the disease, a slower progression of the disease, an improvement in the overall health or well-being of the subject, or by other parameters known in the art that are specific to the particular disease.

Tumor, neoplasia, malignancy or cancer: A neoplasm is an abnormal growth of tissue or cells which results from excessive cell division. Neoplastic growth can produce a tumor. The amount of a tumor in an individual is the “tumor burden” which can be measured as the number, volume, size, or weight of the tumor. A “non-cancerous tissue” is a tissue from the same organ wherein the malignant neoplasm formed, but does not have the characteristic pathology of the neoplasm. Generally, noncancerous tissue appears histologically normal. A “normal tissue” is tissue from an organ, wherein the organ is not affected by cancer or another disease or disorder of that organ. A “cancer-free” subject has not been diagnosed with a cancer of that organ and does not have detectable cancer.

Under conditions sufficient for: A phrase that is used to describe any environment that permits the desired activity. In one example, includes administering a therapeutic agent to a cell or a subject sufficient to allow the desired activity. In particular examples, the desired activity is increasing GITR activity (such as agonizing effector T cells, reducing suppressive activity and lineage stability of T regulatory cells, depleting T regulatory cells, or both).

Unit dose: A physically discrete unit containing a predetermined quantity of an active material calculated to individually or collectively produce a desired effect, such as a therapeutic effect. A single unit dose or a plurality of unit doses can be used to provide the desired effect, such as treatment of a tumor or metastasis. In one example, a unit dose includes a desired amount of one or more agents that enhance or increase GITR expression and/or activity (e.g., a GITR agonist) and one or more CpG ODNs.

Upregulated: When used in reference to the expression of a molecule, such as a (e.g., GITR) gene or a protein, refers to any process which results in an increase in production of a gene product. A gene product can be RNA (such as mRNA, rRNA, tRNA, and structural RNA) or protein. Therefore, upregulation includes processes that increase transcription of a gene or translation of mRNA and thus increase the presence of proteins or nucleic acids, such as a GITR protein or nucleic acid molecule.

Examples of processes that can increase transcription include those that facilitate reduced degradation of a transcription initiation complex, those that increase transcription initiation rate, those that increase transcription elongation rate, those that increase processivity of transcription and those that decrease transcriptional repression. Gene upregulation can include increasing expression above an existing level. Examples of processes that increase translation include those that increase translational initiation, those that increase translational elongation and those that increase mRNA stability. In some examples, a gene expression method, antibody, or other specific binding agent is used to increase expression and/or activity of GITR.

Upregulation includes any detectable statistically significant increase in the production of a gene product, such as a GITR protein. In certain examples, detectable target protein or nucleic acid expression in a cell (such as a tumor cell) increases by at least 20%, at least 30%, at least 40%, at least 50%, at least 60%, at least 70%, at least 75%, at least 80%, at least 90%, at least 95%, at least 96%, at least 97%, at least 98%, at least 99%, at least 100%, at least 150%, at least 200%, at least 250%, at least 300%, at least 350%, at least 400%, at least 500%, or greater than 500% as compared to a control (such an amount of protein or nucleic acid expression detected in a corresponding control cell or sample, such as an untreated tumor cell).

Vector: A nucleic acid molecule as introduced into a host cell, thereby producing a transformed host cell. A vector may include nucleic acid sequences that permit it to replicate in a host cell, such as an origin of replication. A vector may also include one or more selectable marker genes and other genetic elements known in the art. In some examples, a vector includes a promoter and a GITR-encoding nucleic acid molecule.

### Methods of Treating a Tumor

Provided herein are methods for treating a tumor in a subject, such as a mammalian subject (e.g., human, non-human primate, laboratory mammal, or veterinary subject, such as mice, rats, chimpanzees, apes, horses, as well as pets, such as dogs and cats). The subject can be a child or an adult. The methods include intratumoral administration of an effective amount of at least one glucocorticoid-induced TNFR-related protein (GITR) agonist and intratumoral administration of an effective amount of at least one CpG ODN or other TLR9 agonist, thereby treating the tumor in the subject.

In some examples, the at least one GITR agonist and the at least one CpG ODN are administered simultaneously or contemporaneously. In some examples, the at least one GITR agonist and the at least one CpG ODN are administered on at least two different occasions at least twice, such as 2, 3, 4, 5, 6, 7, 8, 9 or 10 times. In some examples, both the GITR agonist and the CpG ODN are administered in multiple doses, at least 1 day, at least 2 days, at least 5 days, at least 7 days at least 14 days, or at least 30 days apart. In some examples, the method further includes administering an effective amount of one or more additional therapeutic agents, such as one or more chemotherapeutic agents, one or more biologic agents, one or more anti-angiogenesis agents, one or more growth inhibitory agents, one or more anti-neoplastic compositions, or combinations thereof. In some examples, the method further includes performing surgery on the subject, for example to remove at least a portion of a tumor.

### GITR Agonists

In some examples, GITR agonists can increase the expression of nucleic acid sequences (such as DNA, cDNA, or mRNAs) and/or proteins. In some examples, the GITR agonist enhances or increases the biological activity of GITR. In some examples, combinations of GITR agonists are used. For example, GITR agonists can be used to enhance or increase the activity of GITR, for example by agonizing effector T cells, reducing the suppressive activity and lineage stability of T regulatory cells, depleting T regulatory cells, enhancing binding of a GITRL to a GITR, or combinations thereof. Such increases are desirable when dowregulation of GITR (or decreased GITR activity) causes, results in, or exacerbates disease, or is undesirable (e.g., increasing the biological activity GITR in a subject with a tumor).

In some examples, an antibody (such as polyclonal antibodies, monoclonal antibodies, chimeric antibodies, camelid antibodies, or humanized antibodies), antibody fragment, antibody conjugate, small organic molecule, small inorganic molecule, large molecule, GITR-encoding nucleic acid molecule, GITR protein, GITR ligand, or combinations thereof, is used. Exemplary GITR agonists target a GITR nucleic acid sequence shown in GenBank Accession No. NM_148902.1, NM_148901.1, NM_004195.2, NM_009400.2, or NM_021985.2 (or sequences having at least 80%, at least 85%, at least 90%, at least 92%, at least 95%, at least 98%, or at least 99% sequence identity to such sequences), or a GITR protein sequence shown in GenBank Accession No. NP_683700.1 (isoform 3 precursor, mature peptide is aa 26-234), NP_683699.1 (isoform 2 precursor, mature peptide is aa 26-255), NP_004186.1 (isoform 1 precursor, mature peptide is aa 26-241), NP_033426.1 (isoform 1 precursor, mature peptide is aa 20-228) or NP_068820.1 (isoform 2 precursor, mature peptide is aa 22-132) (or sequences having at least 80%, at least 85%, at least 90%, at least 92%, at least 95%, at least 98%, or at least 99% sequence identity to such sequences). In some examples, a GITR agonist increases the expression and/or activity of GITR by at least 20%, at least 30%, at least 40%, at least 50%, at least 60%, at least 70%, at least 75%, at least 80%, at least 90%, at least 95%, at least 98%, at least 99%, at least 100%, at least 200%, at least 300%, at least 400%, at least 500% as compared to a control (such as an amount prior to treatment or an amount in the absence of the agonist).

In some examples, a GITR agonist increases GITR activity by at least 20%, at least 30%, at least 40%, at least 50%, at least 60%, at least 70%, at least 75%, at least 80%, at least 90%, at least 95%, at least 96%, at least 97%, at least 98%, or at least 99% (such as a decrease of 40% to 90%, 40% to 80% or 50% to 95%) as compared to a control (such as an amount of GITR activity in the absence of treatment with a GITR agonist or prior to treatment with a GITR agonist). In some embodiments, the disclosed methods include measuring GITR activity.

In some examples, a GITR agonist increases expression of a GITR nucleic acid or protein (e.g., in a tumor cell) by at least 20%, at least 30%, at least 40%, at least 50%, at least 60%, at least 70%, at least 75%, at least 80%, at least 90%, at least 95%, at least 96%, at least 97%, at least 98%, or at least 99% (such as an increase of 40% to 90%, 40% to 80% or 50% to 95%) as compared to a control (such as an amount of GITR activity in the absence of treatment with a GITR agonist or prior to treatment with a GITR agonist). In some embodiments, the disclosed methods include measuring GITR nucleic acid or protein expression.

GITR agonists can be specific for a GITR gene sequence, coding sequence, and/or protein sequence. Exemplary sequences that can be targeted with the disclosed methods and compositions are known (see above for exemplary GenBank® Accession Nos. which provide exemplary nucleic acid and protein sequences). In addition, one skilled in the art will appreciate that variant sequences which retain GITR activity can be targeted. For example, such variants may include encode a protein with one or more deletions, substitutions, or additions (or combinations thereof), such as 1-50 of such changes (such as 1-40, 1-30, 1-20, or 1-10 of such changes). In certain examples, a GITR nucleic acid sequence or protein sequence targeted by the disclosed methods or compositions has at least 80%, at least 85%, at least 90%, at least 95%, at least 97%, at least 98% or at least 99% sequence identity to a GenBank® Accession No. provided herein.

1. Increasing GITR Biological Activity and/or Expression with Antibodies

In one example, the GITR agonist used in combination with one or more CpG ODNs to treat a tumor is an antibody. In certain embodiments, the antibody is a monoclonal antibody, chimeric antibody, human antibody, bispecific antibody or a humanized antibody. In certain examples, the agonist antibody or antibody fragment specifically binds to a mammalian GITR, such as a human GITR. Examples of such antibodies include those that that bind GITR with high specificity. In certain examples, an agonist antibody binds human GITR with a K_{D} of less than 1 nM. In one example, the agonist is an antibody fragment (such as a single chain antibody, Fv, a single-chain Fv (scFv), a Fab, a Fab′, or a (Fab′)₂). Examples of such antibody fragments include those that that bind on GITR with high specificity. In certain examples, the agonist antibody fragment binds human GITR. In certain examples, an agonist antibody fragment binds human GITR with a K_{D} of less than 1 nM.

Examples of GITR agonists and antibodies that can be used include, but are not limited to: TRX518 (a GITR humanized antibody from GITR, Inc.), MK-4166 (GITR agonist antibody from Merck), MK-1248 (from Merck), MEDI1873 (a GITRL-Fc fusion protein from Medimmune), BMS (entity unknown), AMG228 (from Amgen), and INCAGN01876 (from Agenus/Incyte). Further agonist anti-GITR antibodies are described in, e.g., U.S. Pat. Nos. 9,493,572, 9,464,139, 9,309,321, 9,228,016, 9,241,992, 8,709,424, US2017/0145104, US2016/0199487, US2015/0368349, U.S. Pat. No. 9,005,619, WO2017/087678 and WO2017/068186.

In some embodiments, antibodies that agonize GITR activity agonize effector T cells, reduce suppressive activity and lineage stability of T regulatory cells, deplete T regulatory cells, or combinations thereof. In some embodiments, an agonist anti-GITR antibody activates GITR signaling function, for example, by activation of NF-κB response. This activation can be assayed using a system that monitors NF-κB-driven production of a reporter, secreted alkaline phosphatase (SEAP), as described in Example 5 of WO 2017/015623. As described therein, HEK293 cell lines containing an NF-κB-driven SEAP reporter gene (obtained from Invivogen, San Diego, Calif., USA) were stably transfected with GITR and the cell lines were then incubated with titrating doses of anti-GITR antibodies overnight at 37° C. SEAP reporter gene expression at each dose was then quantified in the cell culture supernatant by hydrolysis of a chromogenic substrate by monitoring changes of optical density at 650 nanometers. An increase in SEAP production in this assay over background due to addition of the antibody indicates that the antibody activates GITR signaling function.

In some embodiments, an agonistic GITR antibody binds to GITR with a binding affinity (K_{D}) of less than 50 nM, less than 20 nM, less than 10 nM, or less than 1 nM. In some embodiments, the extent of binding of an agonistic GITR antibody to an unrelated, non-GITR protein is less than about 10% of the binding of the antibody to GITR as measured, e.g., by a radioimmunoassay (RIA). In some embodiments, an agonistic GITR antibody binds to an epitope of GITR that is conserved among GITR from different species. In some examples, an agonistic GITR antibody binds to the same epitope as a human or humanized GITR antibody that binds human GITR.

In some embodiments, an agonistic GITR antibody has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸ M to 10⁻¹³M, e.g., from 10⁻⁹M to 10⁻¹³ M) for GITR.

In some embodiments, a GITR agonistic antibody binds to GITR from multiple species. For example, in some embodiments, an antibody binds to human GITR, and also binds to GITR from at least one mammal selected from mouse, rat, dog, guinea pig, and monkey. In some examples, an antibody binds to GITR from only one species, such as only Homo sapiens.

In some embodiments, an agonist anti-GITR antibody herein may have one or more of the following properties: (a) comprises a GITR binding domain with a K_{D} for GITR of less than 10 nM; (b) binds to both human and cynomolgus monkey GITR; (c) blocks binding between GITR and its ligand GITRL; and (d) costimulates an anti-tumor response while also inhibiting the suppressive effect of T regulatory (Treg) cells.

In one example, the agonistic antibody that binds to GITR is a multispecific antibody, such as a bispecific antibody or a dual variable domain antibody. Nonlimiting exemplary bispecific antibodies include antibodies comprising a first arm comprising a heavy chain/light chain combination that binds a first antigen and a second arm comprising a heavy chain/light chain combination that binds a second antigen. In some embodiments, a bispecific antibody comprises a first arm that binds GITR and a second arm that stimulates immune cell maturation.

In one example, the agonistic antibody that binds to GITR is a single chain antibody, such as a camelid antibody, or comprises a GITR binding domain derived from such antibody. For example, in some embodiments, an anti-GITR antibody may comprise at least one polypeptide that specifically binds GITR, wherein the polypeptide comprises at least one GITR-binding domain comprising three complementarity determining regions (CDRs) derived, for example, from a single domain antibody. In some embodiments, the at least one GITR-binding domain comprises a complementarity determining region 1 (CDR1) comprising the amino acid sequence of SEQ ID NO: 6, a complementarity determining region 2 (CDR2) comprising the amino acid sequence of SEQ ID NO: 7, and a complementarity determining region 3 (CDR3) comprising the amino acid sequence of SEQ ID NO: 8. In some embodiments, the antibodies are polyvalent (or multivalent), and comprise more than one such GITR-binding domain with the above set of CDRs.

In some embodiments, the agonist anti-GITR antibody may comprise at least one polypeptide that comprises at least one GITR-binding domain, wherein the GITR-binding domain in turn comprises the amino acid sequence of SEQ ID NO: 5, or a sequence that is at least 90%, at least 91%, at least 92%, at least 93%, at least 94%, at least 95%, at least 96%, at least 97%, at least 98%, or at least 99% identical to SEQ ID NO: 5. In some embodiments, an anti-GITR antibody comprises two, three, or four GITR-binding domains comprising the amino acid sequence of SEQ ID NO: 5, or a sequence that is at least 90%, at least 91%, at least 92%, at least 93%, at least 94%, at least 95%, at least 96%, at least 97%, at least 98%, or at least 99% identical to SEQ ID NO: 5.

In some embodiments, an agonist anti-GITR antibody comprises a multivalent fusion protein comprising two or more GITR-binding domains fused to a human constant region, such as a human IgG Fc. In some such embodiments, the two or more GITR-binding domains are in tandem (wherein “in tandem” includes configurations in which GITR-binding domains occur sequentially, but optionally with intervening non-Fc sequences between them). In some embodiments, the GITR-binding domains are derived from single domain antibodies and comprise three complementarity determining regions (CDRs). In some embodiments, at least one or all of the GITR-binding domains comprise a complementarity determining region 1 (CDR1) comprising the amino acid sequence of SEQ ID NO: 6, a complementarity determining region 2 (CDR2) comprising the amino acid sequence of SEQ ID NO: 7, and a complementarity determining region 3 (CDR3) comprising the amino acid sequence of SEQ ID NO: 8. In some such embodiments, the human IgG Fc is a human IgG1, IgG2, IgG3, or IgG4. In some embodiments, the multivalent fusion protein comprises two, three, or four GITR-binding domains in tandem, each with the above set of CDRs, fused to a human IgG Fc selected from a human IgG1, IgG2, IgG3, and IgG4 Fc.

In some embodiments, an agonist anti-GITR antibody comprises a multivalent fusion protein comprising two or more GITR-binding domains fused to a human constant region, such as a human IgG Fc, e.g., a human IgG1, IgG2, IgG3, or IgG4 Fc. In some such embodiments, the two or more GITR-binding domains are in tandem. In some such embodiments, at least one or all of the GITR-binding domains comprise the amino acid sequence of SEQ ID NO: 5, or a sequence that is at least 90%, at least 91%, at least 92%, at least 93%, at least 94%, at least 95%, at least 96%, at least 97%, at least 98%, or at least 99% identical to SEQ ID NO: 5. In some such embodiments, the human constant region is a human IgG Fc, such as a human IgG1, IgG2, IgG3, or IgG4 Fc. In some embodiments, the multivalent fusion protein comprises two, three, or four GITR-binding domains in tandem, each comprising the amino acid sequence of SEQ ID NO:19, fused to a human IgG Fc selected from a human IgG1, IgG2, IgG3, and IgG4.

Tetravalent Molecules

In some embodiments, an anti-GITR antibody comprises a tetravalent molecule comprising two copies of a fusion protein having the structure: (GITR-BD)-Linker-(GITR-BD)-Linker-Hinge-Fc, wherein (a) GITR-BD is a GITR binding domain comprising (i) a complementarity determining region 1 (CDR1) comprising the amino acid sequence of SEQ ID NO: 6; a complementarity determining region 2 (CDR2) comprising the amino acid sequence of SEQ ID NO: 7; and a complementarity determining region 3 (CDR3) comprising the amino acid sequence of SEQ ID NO: 8; or comprising (ii) the amino acid sequence of SEQ ID NO: 5, or a sequence that is at least 90%, at least 91%, at least 92%, at least 93%, at least 94%, at least 95%, at least 96%, at least 97%, at least 98%, or at least 99% identical to SEQ ID NO: 5; (b) Linker is a linker polypeptide; (c) Hinge is a polypeptide derived from an immunoglobulin hinge region; and (d) Fc is an immunoglobulin Fc region polypeptide.

In some embodiments in which the fusion protein of a tetravalent molecule comprises a Hinge, the Hinge comprises the amino acid sequence of SEQ ID NO: 15, 16 or 17. For example, the Hinge may comprise a modified IgG1 hinge comprising the amino acid sequence of EPKSSDKTHTCPPC (SEQ ID NO: 15), wherein the Cys220 that forms a disulfide bond with the C-terminal cysteine of the light chain is mutated to serine, e.g., Cys220Ser (C220S). In other embodiments, the fusion protein may comprise a Hinge comprising the amino acid sequence DKTHTCPPC (SEQ ID NO: 16). In some embodiments, the Hinge comprises a hinge from IgG4 that is modified, for example to prevent or reduce strand exchange, e.g., comprising the amino acid sequence ESKYGPPCPPC (SEQ ID NO: 17), in which Ser228 is mutated to Pro (S228P).

In some embodiments in which the fusion protein of a tetravalent molecule comprises a Linker, the Linker comprises an amino acid sequence selected from GG, GGG, and any one of SEQ ID NOs: 18 to 24. In some embodiments, the Linker comprises the amino acid sequence of SEQ ID NO: 18 or 22. In some embodiments, a fusion protein of a tetravalent molecule has a Hinge comprising SEQ ID NO:16 and a Linker comprising SEQ ID NO:18 or 22.

In some embodiments in which the fusion protein of a tetravalent molecule comprises an Fc, the Fc is a human Fc, such as a human IgG1, IgG2, IgG3, or IgG4 Fc. In some embodiments, the Fc comprises an amino acid sequence selected from SEQ ID NOs: 9-14, or a sequence that is at least 90%, at least 91%, at least 92%, at least 93%, at least 94%, at least 95%, at least 96%, at least 97%, at least 98%, or at least 99% identical to one of SEQ ID NOs: 9-14. In some embodiments, the Fc comprises a human IgG1 amino acid sequence such as SEQ ID NO: 9.

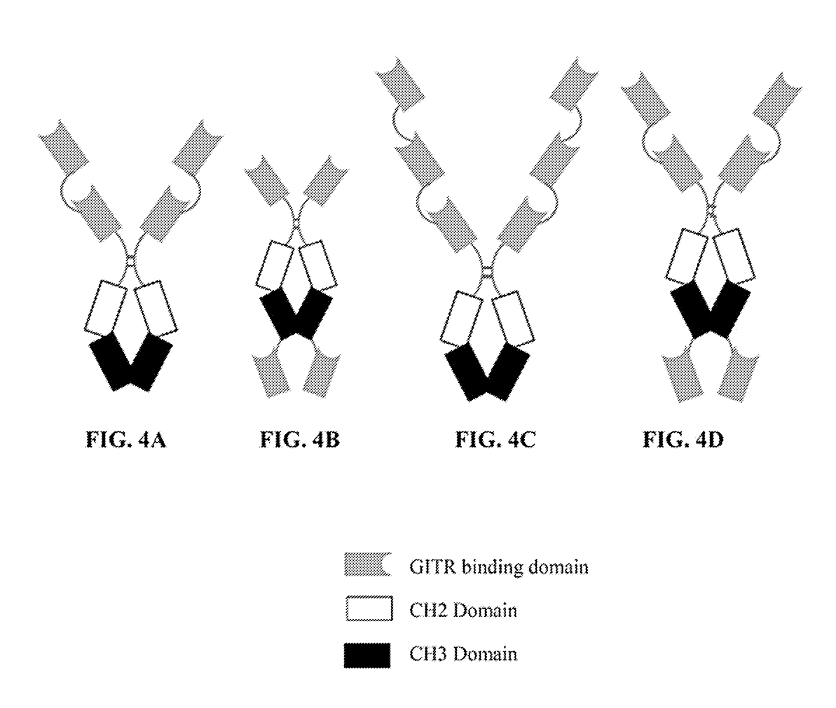

Exemplary tetravalent molecules are shown in FIGS. 4A and 4B. FIG. 4A, for instance, illustrates an exemplary tetravalent molecule in the form of a dimer, with each subunit (polypeptide) having a (GITR-BD)-Linker-(GITR-BD)-Linker-Hinge-Fc architecture, and with each subunit associated with the other by way of a Hinge, and wherein the Fc molecule comprises CH2 and CH3 domains. FIG. 4B, for instance, illustrates an alternative (GITR-BD)-Hinge-Fc-Linker-(GITR-BD) architecture. FIGS. 4C and 4D show hexavalent molecules having structures related to these two tetravalent molecules, i.e., (GITR-BD)-Linker-(GITR-BD)-Linker-(GITR-BD)-Linker-Hinge-Fc in FIG. 4C and (GITR-BD)-Linker-(GITR-BD)-Linker-Hinge-Fc-Linker-(GITR-BD) in FIG. 4D.

In some embodiments, an agonist anti-GITR antibody is a tetravalent molecule comprising two copies of a polypeptide comprising the amino acid sequence of SEQ ID NO: 4 or a sequence that is at least 90%, at least 91%, at least 92%, at least 93%, at least 94%, at least 95%, at least 96%, at least 97%, at least 98%, or at least 99% identical to SEQ ID NO: 4.

Fc Regions

In any of the foregoing embodiments, an Fc may comprise an amino acid sequence selected from SEQ ID NOs: 9-14, or a sequence that is at least 90%, at least 91%, at least 92%, at least 93%, at least 94%, at least 95%, at least 96%, at least 97%, at least 98%, or at least 99% identical to one of SEQ ID NOs: 9-14. In some embodiments, the Fc comprises a human IgG1 amino acid sequence such as SEQ ID NO: 9. In some embodiments, the Fc comprises the amino acid sequence of SEQ ID NO: 9, but where position Asn297 (boxed in the sequence shown in the sequence table) is modified to inhibit fucosylation. In some embodiments, the Fc is a human IgG1 Fc, but where one or more of positions Leu235, Leu236, and Gly237 have been modified to other amino acids (boxed in the sequence shown in the sequence table). In some embodiments, the Fc comprises a human IgG1 Fc lacking Lys447. In some embodiments, the Fc is a human IgG1 Fc that lacks an amino acid at one or more of Glu233, Leu234, or Leu235, as provided, for example, in SEQ ID NO: 10. In some embodiments, the Fc comprises a human IgG2 Fc, e.g. SEQ ID NO: 11. In some embodiments, the Fc comprises a human IgG2 Fc that is modified, for example mutated at Asn297 (boxed in the sequence table) or that lacks Lys447. In some embodiments, the Fc comprises a human IgG3 Fc, e.g. SEQ ID NO: 12. In some embodiments, the Fc comprises a human IgG3 Fc that is modified, for example, mutated at Asn297, contains an Arg to His substitution at position 435 (both boxed in the sequence table), or that lacks Lys447. In some embodiments, the Fc comprises a human IgG4 Fc, e.g. SEQ ID NO: 13 or 14. In some embodiments, the Fc comprises a human IgG4 Fc that is modified, for example mutated at position Leu235 or Asn297 (both boxed in the sequence table), or that lacks Lys447.

In some embodiments, the human IgG Fc region is modified to enhance FcRn binding. Examples of Fc mutations that may enhance binding to FcRn are Met252Tyr, Ser254Thr, Thr256Glu (M252Y, S254T, T256E, respectively) (Kabat numbering, Dall'Acqua et al 2006, J. Biol Chem, 281(33) 23514-24), Met428Leu and Asn434Ser (M428L, N434S) (Zalevsky et al 2010 Nature Biotech, 28(2) 157-9), or Met252Ile, Thr256Asp, Met428Leu (M252I, T256D, M428L, respectively), (EU index of Kabat et al 1991 Sequences of Proteins of Immunological Interest).

In some embodiments, a mutated Fc may also include the following substitutions: Met252Tyr and Met428Leu (M252Y, M428L) using the Kabat numbering system.

In some embodiments, the human IgG Fc region may be modified to alter antibody-dependent cellular cytotoxicity (ADCC) and/or complement-dependent cytotoxicity (CDC), e.g., the amino acid modifications described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1):1-11. Examples of mutations that may enhance ADCC include modification at Ser239 and Ile332, for example Ser239Asp and Ile332Glu (S239D, 1332E). Examples of mutations that may enhance CDC include modifications at Lys326 and Glu333. In some embodiments, the Fc region is modified at one or both of these positions, for example Lys326Ala and/or Glu333Ala (K326A and E333A) using the Kabat numbering system.

In some embodiments, the human IgG Fc region may be modified to induce heterodimerization. For example, having an amino acid modification within the CH3 domain at Thr366, which when replaced with a more bulky amino acid, e.g., Typ (T366W), is able to preferentially pair with a second CH3 domain having amino acid modifications to less bulky amino acids at positions Thr366, Leu368, and Tyr407, e.g., Ser, Ala and Val, respectively (T366S/L368A/Y407V). Further CH3 domain modifications, for example, can include changing Ser354 to Cys (S354C) and Y349 to Cys (Y349C) on opposite CH3 domains (Reviewed in Carter, 2001 Journal of Immunological Methods, 248: 7-15).

In some embodiments, the human IgG Fc region is modified to prevent or reduce dimerization of Fc domains. For example, residue Thr366 can be substituted with a charged residue, e.g. Thr366Lys, Thr366Arg, Thr366Asp, or Thr366Glu (T366K, T366R, T366D, or T366E, respectively), which may in some cases prevent CH3-CH3 dimerization.

In some embodiments, the Fc region may be altered at one or more of the following positions to reduce Fc receptor binding: Leu 234 (L234), Leu235 (L235), Asp265 (D265), Asp270 (D270), Ser298 (S298), Asn297 (N297), Asn325 (N325) or Ala327 (A327). For example, Leu 234Ala (L234A), Leu235Ala (L235A), Asp265Asn (D265N), Asp270Asn (D270N), Ser298Asn (S298N), Asn297Ala (N297A), Asn325Glu (N325E) or Ala327Ser (A327S). In some embodiments, modifications within the Fc region may reduce binding to Fc-receptor-gamma receptors while having minimal impact on binding to the neonatal Fc receptor (FcRn).

Sequence Table

| SEQ | | |
|---|---|---|
| ID | | |
| NO | Description | Sequence |
| 4 | GITR binding | EVQLLESGGGEVQPGGSLRLSCAASGSVFSIDAMGWYRQAPGKQRELVA |
| | polypeptide | VLSGISSAKYAASAPGRFTISRDNAKNTVYLQMSSLRAEDTAVYYCYAD |
| | | VSTGWGRDAHGYWGQGTLVTVKPGGSGGSEVQLLESGGGEVQPGGSLRL |
| | | SCAASGSVFSIDAMGWYRQAPGKQRELVAVLSGISSAKYAASAPGRFTI |
| | | SRDNAKNTVYLQMSSLRAEDTAVYYCYADVSTGWGRDAHGYWGQGTLVT |
| | | VKPGGGGDKTHTCPPCPAPELLGGPSVFLFPPKPKDTLMISRTPEVTCV |
| | | VVDVSHEDPEVKFNWYVDGVEVHNAKTKPREEQYNSTYRVVSVLTVLHQ |
| | | DWLNGKEYKCKVSNKALPAPIEKTISKAKGQPREPQVYTLPPSRDELTK |
| | | NQVSLTCLVKGFYPSDIAVEWESNGQPENNYKTTPPVLDSDGSFFLYSK |
| | | LTVDKSRWQQGNVFSCSVMHEALHNHYTQKSLSLSPGK |
| 5 | GITR binding | EVQLLESGGGEVQPGGSLRLSCAASGSVFSIDAMGWYRQAPGKQRELVA |
| | domain | VLSGISSAKYAASAPGRFTISRDNAKNTVYLQMSSLRAEDTAVYYCYAD |
| | | VSTGWGRDAHGYWGQGTLVTV |
| 6 | CDR1 | SGSVFSIDAM |
| 7 | CDR2 | LSGISSAK |
| 8 | CDR3 | YADVSTGWGRDAHGYW |
| 9 | Human IgG1 Fc | 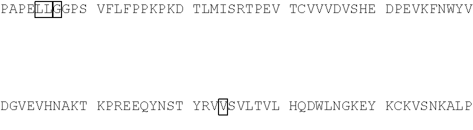 |
| | | APIEKTISKA KGQPREPQVY TLPPSRDELT KNQVSLTCLV KGFYPSDIAV |
| | | EWESNGQPEN NYKTTPPVLD SDGSFFLYSK LTVDKSRWQQ GNVFSCSVMH |
| | | EALHNHYTQK SLSLSPGK |
| 10 | Human IgG1 Fc | PAPGGPSVFL FPPKPKDTLM ISRTPEVTCV VVDVSHEDPE VKFNWYVDGV |
| | deletion | EVHNAKTKPR EEQYNSTYRV VSVLTVLHQD WLNGKEYKCK VSNKALPAPI |
| | mutant at | EKTISKAKGQ PREPQVYTLP PSRDELTKNQ VSLTCLVKGF YPSDIAVEWE |
| | E233, L234, | SNGQPENNYK TTPPVLDSDG SFFLYSKLTV DKSRWQQGNV FSCSVMHEAL |
| | L235 | HNHYTQKSLS LSPGK |
| 11 | Human IgG2 Fc | PAPPVAGPSV FLFPPKPKDT LMISRTPEVT CVVVDVSHED PEVQFNWYVD |
| | | 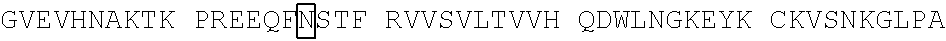 |
| | | PIEKTISKTK GQPREPQVYT LPPSREEMTK NQVSLTCLVK GFYPSDISVE |
| | | WESNGQPENN YKTTPPMLDS DGSFFLYSKL TVDKSRWQQG NVFSCSVMHE |
| | | ALHNHYTQKS LSLSPGK |
| 12 | Human IgG3 Fc | PAPELLGGPS VFLFPPKPKD TLMISRTPEV TCVVVDVSHE DPEVQFKWYV |
| | | 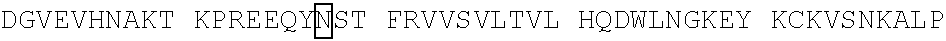 |
| | | APIEKTISKT KGQPREPQVY TLPPSREEMT KNQVSLTCLV KGFYPSDIAV |
| | | EWESSGQPEN NYNTTPPMLD SDGSFFLYSK LTVDKSRWQQ GNIFSCSVMH |
| | | 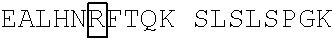 |
| 13 | Human IgG4 Fc | 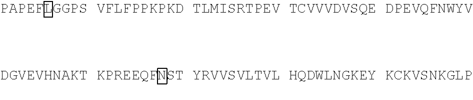 |
| | | SSIEKTISKA KGQPREPQVY TLPPSQEEMT KNQVSLTCLV KGFYPSDIAV |
| | | EWESNGQPEN NYKTTPPVLD SDGSFFLYSR LTVDKSRWQE GNVFSCSVMH |
| | | EALHNHYTQK SLSLSLGK |
| 14 | Human IgG4 Fc | PAPELLGGPS VFLFPPKPKD TLMISRTPEV TCVVVDVSQE DPEVQFNWYV |
| | | 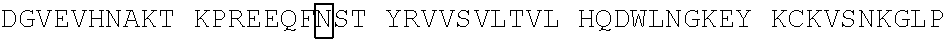 |
| | | SSIEKTISKA KGQPREPQVY TLPPSQEEMT KNQVSLTCLV KGFYPSDIAV |
| | | EWESNGQPEN NYKTTPPVLD SDGSFFLYSR LTVDKSRWQE GNVFSCSVMH |
| | | EALHNHYTQK SLSLSLGK |
| 15 | IgG hinge | EPKSSDKTHTCPPC |
| | region | |
| 16 | IgG hinge | DKTHTCPPC |
| | region | |
| 17 | IgG hinge | ESKYGPPCPPC |
| | region | |
| 18 | Linker | GGSGGS |
| | sequence | |
| 19 | Linker | GGSGGSGGS |
| | sequence | |
| 20 | Linker | GGSGGSGGSGGS |
| | sequence | |
| 21 | Linker | GGSGGSGGSGGSGGS |
| | sequence | |
| 22 | Linker | GGGG |
| | sequence | |
| 23 | Linker | GGGGG |
| | sequence | |
| 24 | Linker | GGGGGG |
| | sequence | |

a. Humanized Antibodies

In one example, the agonistic antibody that binds to GITR is a humanized antibody. Humanized antibodies are useful as therapeutic molecules because humanized antibodies reduce or eliminate the human immune response to non-human antibodies (such as the human anti-mouse antibody (HAMA) response), which can result in an immune response to an antibody therapeutic, and decreased effectiveness of the therapeutic. An antibody may be humanized by any method. Nonlimiting exemplary methods of humanization include methods described, e.g., in U.S. Pat. Nos. 5,530,101; 5,585,089; 5,693,761; 5,693,762; 6,180,370; Jones et al., Nature 321: 522-525 (1986); Riechmann et al., Nature 332: 323-27 (1988); Verhoeyen et al., Science 239: 1534-36 (1988); and U.S. Publication No. US 2009/0136500.

A humanized antibody is an antibody in which at least one amino acid in a framework region of a non-human variable region has been replaced with the amino acid from the corresponding location in a human framework region. In some embodiments, at least two, at least three, at least four, at least five, at least six, at least seven, at least eight, at least nine, at least 10, at least 11, at least 12, at least 15, or at least 20 amino acids in the framework regions of a non-human variable region are replaced with an amino acid from one or more corresponding locations in one or more human framework regions.

In some embodiments, some of the corresponding human amino acids used for substitution are from the framework regions of different human immunoglobulin genes. That is, in some such embodiments, one or more of the non-human amino acids may be replaced with corresponding amino acids from a human framework region of a first human antibody or encoded by a first human immunoglobulin gene, one or more of the non-human amino acids may be replaced with corresponding amino acids from a human framework region of a second human antibody or encoded by a second human immunoglobulin gene, one or more of the non-human amino acids may be replaced with corresponding amino acids from a human framework region of a third human antibody or encoded by a third human immunoglobulin gene, etc. Further, in some embodiments, all of the corresponding human amino acids being used for substitution in a single framework region, for example, FR2, need not be from the same human framework. In some embodiments, however, all of the corresponding human amino acids being used for substitution are from the same human antibody or encoded by the same human immunoglobulin gene.

In some embodiments, an antibody is humanized by replacing one or more entire framework regions with corresponding human framework regions. In some embodiments, a human framework region is selected that has the highest level of homology to the non-human framework region being replaced. In some embodiments, such a humanized antibody is a CDR-grafted antibody.

In some embodiments, following CDR-grafting, one or more framework amino acids are changed back to the corresponding amino acid in a mouse framework region. Such “back mutations” are made, in some embodiments, to retain one or more mouse framework amino acids that appear to contribute to the structure of one or more of the CDRs and/or that may be involved in antigen contacts and/or appear to be involved in the overall structural integrity of the antibody. In some embodiments, ten or fewer, nine or fewer, eight or fewer, seven or fewer, six or fewer, five or fewer, four or fewer, three or fewer, two or fewer, one, or zero back mutations are made to the framework regions of an antibody following CDR grafting.

In some embodiments, a humanized antibody also includes a human heavy chain constant region and/or a human light chain constant region.

b. Chimeric Antibodies

In one example, the GITR-specific agonistic antibody is a chimeric antibody. In some embodiments, the GITR-specific agonistic antibody includes at least one non-human variable region and at least one human constant region. In some such embodiments, all of the variable regions of the GITR-specific agonistic antibody are non-human variable regions, and all of the constant regions of the GITR-specific agonistic antibody are human constant regions. In some embodiments, one or more variable regions of a chimeric antibody are mouse variable regions. The human constant region of a chimeric antibody need not be of the same isotype as the non-human constant region, if any, it replaces. Chimeric antibodies are discussed, e.g., in U.S. Pat. No. 4,816,567; and Morrison et al. Proc. Natl. Acad. Sci. USA 81: 6851-55 (1984).

c. Human Antibodies

In one example, the GITR agonistic antibody is a human antibody. Human antibodies can be made by any suitable method. Nonlimiting exemplary methods include making human antibodies in transgenic mice that comprise human immunoglobulin loci. See, e.g., Jakobovits et al., Proc. Natl. Acad. Sci. USA 90: 2551-55 (1993); Jakobovits et al., Nature 362: 255-8 (1993); Lonberg et al., Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806. Nonlimiting exemplary methods also include making human antibodies using phage display libraries. See, e.g., Hoogenboom et al., J. Mol. Biol. 227: 381-8 (1992); Marks et al., J. Mol. Biol. 222: 581-97 (1991); and PCT Publication No. WO 99/10494.

d. Human Antibody Constant Regions

In some embodiments, a humanized, chimeric, or human GITR agonistic antibody described herein includes one or more human constant regions. In some embodiments, the human heavy chain constant region is of an isotype selected from IgA, IgG, and IgD. In some embodiments, the human light chain constant region is of an isotype selected from κ and λ. In some embodiments, a GITR agonistic antibody includes a human IgG constant region, for example, human IgG1, IgG2, IgG3, or IgG4. In some embodiments, an antibody or Fc fusion partner comprises a C237S mutation, for example, in an IgG1 constant region. In some embodiments, a GITR agonistic antibody includes a human IgG2 heavy chain constant region. In some such embodiments, the IgG2 constant region includes a P331S mutation, as described in U.S. Pat. No. 6,900,292. In some embodiments, a GITR agonistic antibody includes a human IgG4 heavy chain constant region. In some such embodiments, a GITR agonistic antibody includes an S241P mutation in the human IgG4 constant region. See, e.g., Angal et al. Mol. Immunol. 30(1): 105-108 (1993). In some embodiments, a GITR agonistic antibody includes a human IgG4 constant region and a human κ light chain.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. Typically, antibodies comprising human IgG1 or IgG3 heavy chains have effector function.

In some embodiments, effector function is not desirable. For example, in some embodiments, effector function may not be desirable in treatments of tumors. In some such embodiments, a human IgG4 or IgG2 heavy chain constant region is selected or engineered. In some embodiments, an IgG4 constant region includes an S241P mutation.

e. Antibody Conjugates

In some embodiments, an agonistic GITR antibody is conjugated to a label. A label is a moiety that facilitates detection of the antibody and/or facilitates detection of a molecule to which the antibody binds. Nonlimiting exemplary labels include, but are not limited to, radioisotopes, fluorescent groups, enzymatic groups, chemiluminescent groups, biotin, epitope tags, metal-binding tags, etc. In some embodiments, a label is conjugated to an antibody using chemical methods in vitro. Nonlimiting exemplary chemical methods of conjugation are known in the art, and include services, methods and/or reagents commercially available from, e.g., Thermo Scientific Life Science Research Produces (formerly Pierce; Rockford, Ill.), Prozyme (Hayward, Calif.), SACRI Antibody Services (Calgary, Canada), AbD Serotec (Raleigh, N.C.), etc. In some embodiments, when a label is a polypeptide, the label can be expressed from the same expression vector with at least one antibody chain to produce a polypeptide comprising the label fused to an antibody chain.

In some embodiments, an agonistic GITR antibody that specifically binds and kills regulatory T cells but not T effector cells is conjugated to a compound, such as a toxin or drug, for example via a linker. Such compounds are sometimes referred to antibody-drug conjugates (ADCs). Linkers can be clearable or non-cleavable. Exemplary toxins include but are not limited to, cytotoxins, such as a chemotherapeutic agent, an agent that damages DNA, an agent that interferes with tubulin polymerization, or a radionuclide (examples of such cytotoxins are provided elsewhere in this disclosure). In one example, the toxin is a plant-derived protein toxin (such as gelonin, ricin, abrin, or pokeweed antiviral protein), or a bacterial toxin (such as Pseudomonas exotoxin and Diphtheria toxin).

f. Production of Antibodies

In some examples, a GITR antibody is produced using routine methods to generate polyclonal or monoclonal antibodies.

In some embodiments, a GITR antibody described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

In some examples, a GITR antibody described herein is produced from a nucleic acid molecule, wherein the nucleic acid molecule encodes one or more chains of an antibody described herein. In some examples, a nucleic acid molecule comprises a polynucleotide that encodes a heavy chain or a light chain of a GITR antibody described herein. In some examples, a nucleic acid molecule includes both a polynucleotide that encodes a heavy chain and a polynucleotide that encodes a light chain, of a GITR antibody described herein. In some embodiments, a first nucleic acid molecule comprises a first polynucleotide that encodes a heavy chain and a second nucleic acid molecule comprises a second polynucleotide that encodes a light chain. In some such examples, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some examples, a polynucleotide encoding a heavy chain or light chain of a GITR antibody comprises a nucleotide sequence that encodes a leader sequence, which, when translated, is located at the N-terminus of the heavy chain or light chain. As discussed above, the leader sequence may be the native heavy or light chain leader sequence, or may be another heterologous leader sequence.

Nucleic acid molecules may be constructed using recombinant DNA techniques. In some embodiments, a nucleic acid molecule is an expression vector that is suitable for expression in a selected host cell (such as those vectors described below). Heavy chains and/or light chains of a GITR antibody can be expressed in prokaryotic cells, such as bacterial cells; or in eukaryotic cells, such as fungal cells (such as yeast), plant cells, insect cells, and mammalian cells. In some examples, one or more polypeptides may be produced in vivo in an animal that has been engineered or transfected with one or more nucleic acid molecules encoding the polypeptides, according to any suitable method.

A GITR antibody may be purified by any suitable method, such as by the use of affinity matrices or hydrophobic interaction chromatography. Suitable affinity ligands include the antigen and/or epitope to which the antibody binds, and ligands that bind antibody constant regions. For example, a Protein A, Protein G, Protein A/G, or an antibody affinity column may be used to bind the constant region and to purify an antibody. In some embodiments, hydrophobic interactive chromatography, for example, a butyl or phenyl column, is also used for purifying some polypeptides.

2. Increasing GITR Biological Activity and/or Expression with Nucleic Acids

In one example, GITR biological activity is enhanced or increased by use of a nucleic acid molecule encoding GITR. For example, GITR-encoding nucleic acid molecules can be used to increase gene expression of GITR, for example where upregulation is desirable (e.g., increasing expression of GITR in or in the area of a tumor).

In some examples, GITR-encoding nucleic acid molecules increase expression of GITR, for example by at least 20%, at least 30%, at least 40%, at least 50%, at least 60%, at least 75%, at least 90%, at least 100%, at least 200%, at least 300%, at least 400%, or at least 500%. In some examples, the GITR-encoding nucleic acid molecule increases the biological activity of GITR by at least 20%, at least 30%, at least 40%, at least 50%, at least 60%, at least 70%, at least 75%, at least 80%, at least 90%, at least 95%, at least 98%, at least 99%, at least 100%, at least 200%, at least 300%, at least 400%, or at least 500% for example relative to such activity in the absence of the GITR-encoding nucleic acid molecule. Exemplary GITR-encoding nucleic acid molecules are provided in GenBank Accession Nos. NM_148902.1, NM_148901.1, NM_004195.2, NM_009400.2, and NM_021985.2 (or a sequence having at least 70%, at least 80%, at least 85%, at least 90%, at least 92%, at least 95%, at least 98%, or at least 99% sequence identity to such sequences).

Nucleic acid molecules encoding GITR can be introduced into the cells of a subject (such as tumor cells) using routine methods, such as by using recombinant vectors (e.g., plasmid or viral vector, for example by placing the GITR-encoding nucleic acid molecule under the control of a suitable promoter) or by using naked nucleic acids or nucleic acid complexes (non-viral methods, such as liposome encapsulated RNA). Exemplary vectors include, but are not limited to, DNA vectors, phage vectors, viral vectors, and retroviral vectors. Exemplary viral vectors that can be used include, but are not limited to: pox viruses, recombinant vacciniavirus, retroviruses (such as lentivirus), replication-deficient adenovirus strains, adenovirus vectors, adeno-associated virus (AAV), herpes virus, or poliovirus. Exemplary promoters include constitutive or inducible promoters, such as a tissue-specific promoter.

Introduction of one or more nucleic acids into a desired host cell can be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

In some examples, the GITR-encoding nucleic acid molecule is only introduced into certain cells or tissues. For example, introducing GITR-encoding nucleic acid molecules into only the tumor may be sufficient. However, in some instances, it may be more therapeutically effective and simple to treat all of the subject's cells, or more broadly disseminate the GITR-encoding nucleic acid molecules for example by intravascular administration. In some examples, the GITR-encoding nucleic acid molecule(s) are administered directly to a subject, for example by injection (intradermal, intramuscular, iv, intratumoral, or subcutaneous), topical administration, oral administration, inhalation, infusion, deposition, or implantation.

3. Increasing GITR Biological Activity with GITR Proteins

In one example, GITR biological activity is enhanced or increased by use of an GITR protein. For example, GITR proteins can be used to increase GITR activity, for example where upregulation is desirable (e.g., increasing activity of GITR in the area of a tumor).

Exemplary GITR proteins that can be used to increase GITR activity include those having a GITR protein sequence shown in GenBank® Accession No. NP_683700.1 (isoform 3 precursor, mature peptide is aa 26-234), NP_683699.1 (isoform 2 precursor, mature peptide is aa 26-255), NP_004186.1 (isoform 1 precursor, mature peptide is aa 26-241), NP_033426.1 (isoform 1 precursor, mature peptide is aa 20-228) or NP_068820.1 (isoform 2 precursor, mature peptide is aa 22-132), or a GITR sequence having at least 80%, at least 85%, at least 90%, at least 92%, at least 95%, at least 98%, or at least 99% sequence identity to such sequences.

In some examples, administration of a GITR protein increases the activity of GITR by at least 20%, at least 30%, at least 40%, at least 50%, at least 60%, at least 70%, at least 75%, at least 80%, at least 90%, at least 95%, at least 98%, at least 99%, at least 100%, at least 200%, at least 300%, at least 400%, at least 500% as compared to a control (such as an amount prior to treatment or an amount in the absence of the protein).

### CpG ODNs

Exemplary CpG ODNs include class B ODNs, such as the human CpG sequence shown in SEQ ID NO: 1 (5′-tcgtcgttttgtcgttttgtcgtt-3′ with bases that are phosphorothioate) and the murine CpG ODN sequence shown in SEQ ID NO: 2 (5′-tccatgacgttcctgacgtt-3′ with bases that are phosphorothioate). In one example, the CpG ODN has the sequence 5′-TGACTGTGAACGTTCGAGATGA-3′ SEQ ID NO: 3 (ISS 1018 from Dynavax).

### Administration and Dosages

The disclosed methods include providing a therapeutically effective amount of one or more GITR agonists and one or more CpG ODNs or other TLR9 agonists, for example in combination with therapeutically effective amounts of other therapies, to a subject having a tumor. The one or more GITR agonists and one or more CpG ODNs (or other TLR9 agonists) are administered intratumorally, for example, simultaneously or contemporaneously. It is shown herein that intratumoral administration provides an unexpected and superior result over systemic administration. The dose required may vary from subject to subject depending on the species, age, weight and general condition of the subject, the particular therapeutic agent being used and its mode of administration. In some examples, the disclosed methods further include providing surgery and/or other therapy to the subject in combination with the one or more GITR agonists and one or more CpG ODNs (or other TLR9 agonists), for example, sequentially, substantially simultaneously, or simultaneously.

Administration can be accomplished by single or multiple doses. In some examples, treatment can involve daily or multi-daily or less than daily (such as weekly or monthly etc.) doses of one or more therapeutic agents provided herein over a period of a few days to months, or even years. For example, a therapeutically effective amount of one or more GITR agonists can be administered in a single dose, twice daily, weekly, or in several doses, for example daily, or during a course of treatment. For example, a therapeutically effective amount of one or more CpG ODNs (or other TLR9 agonists) can be administered in a single dose, twice daily, weekly, or in several doses, for example daily, or during a course of treatment. In a particular non-limiting example, treatment involves once a daily dose or twice daily dose of one or more GITR agonists and one or more CpG ODNs (or other TLR9 agonists). In some examples, the one or more GITR agonists and one or more CpG ODNs (or other TLR9 agonists) are administered to the subject once a month, less than once a month, such as, for example, every two months, every three months, or every six months. In other examples, the one or more GITR agonists and one or more CpG ODNs (or other TLR9 agonists) are administered more than once a month, such as, for example, every two weeks, every week, twice per week, three times per week, daily, or multiple times per day. Thus, in some examples, the disclosed methods include at least two separate intratumoral administrations of an effective amount of at least one GITR agonist and at least two separate intratumoral administrations of an effective amount of at least one CpG ODN.

The one or more GITR agonists and/or the one or more CpG ODNs (or other TLR9 agonists) can be used in the form of a solid, a solution, an emulsion, a dispersion, a micelle, a liposome, and the like, for example in admixture with an organic or inorganic carrier or excipient suitable for intratumoral applications. The one or more GITR agonists and/or the one or more CpG ODNs (or other TLR9 agonists) may be compounded, for example, with non-toxic, pharmaceutically acceptable carriers for pellets, solutions, emulsions, suspensions and any other suitable for use. The pharmaceutically acceptable carriers (vehicles) useful in this disclosure are conventional. Remington's Pharmaceutical Sciences, by E. W. Martin, Mack Publishing Co., Easton, Pa., 15th Edition (1975), describes compositions and formulations suitable for pharmaceutical delivery of one or more therapeutic agents. Exemplary carriers include glucose, lactose, gum acacia, gelatin, manitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening, and coloring agents and perfumes may be used.

Intratumoral administration is generally achieved by injection. Injectables can be prepared in conventional forms, either as liquid solutions or suspensions, solid forms suitable for solution of suspension in liquid prior to injection, or as emulsions. Injection solutions and suspensions can be prepared from sterile powders, granules, and tablets. Preparations for intratumoral administration (such as a sterile injectable suspension) include sterile aqueous or non-aqueous solutions, suspensions, and emulsions. A suspension can be formulated according to known methods using suitable dispersing or wetting agents and suspending agents. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered saline, dextrose, water, glycerol, ethanol, and combinations thereof. Exemplary vehicles include sodium chloride solution, Ringer's dextrose, dextrose and sodium chloride, lactated Ringer's, or fixed oils. Preservatives and other additives may also be present such as, for example, antimicrobials, anti-oxidants, chelating agents, buffers, and inert gases and the like. In one example, a sterile injectable preparation that includes one or more GITR agonists and/or one or more CpG ODNs (or other TLR9 agonists) is a sterile injectable solution or suspension in a non-toxic parenterally-acceptable diluent or solvent, for example, as a solution in 1,3-butanediol. A sterile, fixed oil can be employed as a solvent or suspending medium. For this purpose any bland fixed oil may be employed including synthetic mono- or diglycerides, fatty acids, naturally occurring vegetable oils like sesame oil, coconut oil, peanut oil, cottonseed oil, etc., or synthetic fatty vehicles like ethyl oleate or the like.

In one example, a compositions that includes one or more GITR agonists and/or one or more CpG ODNs (or other TLR9 agonists) is formulated for injection, including intratumoral administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. The compositions can also be formulated, in various embodiments, into sustained release microcapsules, such as with biodegradable or non-biodegradable polymers. A nonlimiting exemplary biodegradable formulation includes poly lactic acid-glycolic acid polymer. A nonlimiting exemplary non-biodegradable formulation includes a polyglycerin fatty acid ester. Certain methods of making such formulations are described, for example, in EP 1 125 584 A1.

The one or more GITR agonists and/or one or more CpG ODNs (or other TLR9 agonists), alone or in combination with other therapies, can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines

In some embodiments, a nucleic acid molecule (such as a GITR-encoding nucleic acid molecule or CpG ODN) is delivered using gene therapy. As a nonlimiting example, a nucleic acid molecule may be coated onto gold microparticles and delivered by a particle bombardment device, or “gene gun,” e.g., as described in the literature (see, e.g., Tang et al., Nature 356:152-154 (1992)).

Therapeutic dosages of one or more GITR agonists and/or one or more CpG ODNs (or other TLR9 agonists) can be determined by a skilled clinician. Other therapeutic agents, for example agents for treating a tumor, also are suitable for administration in combination with one or more GITR agonists and one or more CpG ODNs (or other TLR9 agonists).

In some embodiments, the dose of a GITR agonistic antibody, antibody fragment, or antibody conjugate is less than used for systemic administration, such as at least 2-fold, at least 5-fold, at least 10-fold, at least 20-fold, at least 50-fold, at least 75-fold, or at least 100-fold less than used for systemic administration. In some examples, the dose of a GITR agonistic antibody is no more than 1 mg/kg, no more than 0.3 mg/kg, no more than 0.25 mg/kg, no more than 0.03 mg/kg, no more than 0.01 mg/kg. In some embodiments, the dose of a GITR agonistic antibody is at least 0.01 mg/kg, at least 0.03 mg/kg, at least 0.25 mg/kg, at least 0.3 mg/kg, or at least 1 mg/kg. In some embodiments, the dose of a GITR agonistic antibody is about 0.001 mg/kg to about 5 mg/kg, such as about 0.01 mg/kg to about 5 mg/kg, about 0.01 mg/kg to about 1 mg/kg, or about 0.03 mg/kg to about 1 mg/kg. In some examples, the dose of antibody is about 0.01 mg/kg, about 0.02 mg/kg, about 0.03 mg/kg, about 0.1 mg/kg, about 0.2 mg/kg, about 0.25 mg/kg, about 0.3 mg/kg, about 0.5 mg/kg, about 0.8 mg/kg, or about 1 mg/kg.

In some embodiments, the dose of a GITR protein is at least 0.2 μg, at least 0.5 μg, at least 1 μg, at least 2 μg, at least 5 μg, or at least 10 μg. In some examples, the effective amount of the at least one GITR protein is no more than 100 μg, no more than 50 μg, no more than 25 μg, no more than 10 μg, or no more than 2 μg, for example 1 μg to 100 μg.

In some examples, the dose of the at least one CpG ODN is at least 0.05 mg, at least 0.3 mg, at least 1 mg, at least 3 mg, at least 6 mg, at least 18 mg, or at least 20 mg, such as 0.05 mg, 0.1 mg, 0.2 mg, 0.3 mg, 1 mg, 3 mg, 6 mg, 14 mg, or 18 mg. In some examples, the dose of the at least one CpG ODN is no more than 20 mg, no more than 10 mg, no more than 1 mg, no more than 0.3 mg, or no more than 0.05 mg, such as 005 mg to 20 mg.

In some embodiments, the dose of an agonistic small molecule is at least 1 mg/kg, at least 10 mg/kg, at least 50 mg/kg, at least 100 mg/kg, at least 500 mg/kg, or at least 1000 mg/kg, such as about 1 mg/kg to about 1000 mg/kg, about 10 mg/kg to about 200 mg/kg, about 10 mg/kg to about 100 mg/kg, or about 100 mg/kg to about 500 mg/kg. In some examples, the dose of an agonistic small molecule is about 1 mg/kg, about 2 mg/kg, about 4 mg/kg, about 5 mg/kg, about 6 mg/kg, about 8 mg/kg, about 10 mg/kg, about 15 mg/kg, about 20 mg/kg, or about 25 mg/kg. In other embodiments, the dose of an agonistic small molecule is at least 1 mg/m², at least 10 mg/m², at least 50 mg/m², at least 100 mg/m², or at least 500 mg/m², such as about 50 mg/m² to about 500 mg/m², such as about 50 mg/m² to about 400 mg/m², about 100 mg/m² to about 400 mg/m², or about 250 mg/m² to about 400 mg/m². In some examples, the dose is about 50 mg/m², about 100 mg/m², about 150 mg/m², about 200 mg/m², about 250 mg/m², about 300 mg/m², about 400 mg/m², or about 500 mg/m².

In some examples, the dose of a GITR-encoding nucleic acid (alone or as part of a plasmid or vector) is about 1 mg to about 1000 mg, about 10 mg to about 500 mg, about 1 mg to about 10 mg, or about 50 mg to about 100 mg. In some examples, the dose of a GITR-encoding nucleic acid is about 1 mg, about 2 mg, about 2.5 mg, about 3 mg, about 5 mg, about 10 mg, about 50 mg, about 100 mg, about 250 mg, about 500 mg or about 1000 mg. In some embodiments, the dose of a GITR-encoding nucleic acid is about 1 mg/kg to about 100 mg/kg, about 1 mg/kg to about 10 mg/kg, about 5 mg/kg to about 500 mg/kg, about 10 mg/kg to about 100 mg/kg, or about 25 to about 50 mg/kg. In some examples, the dose of a GITR-encoding nucleic acid is about 1 mg/kg, 2 mg/kg, 3 mg/kg, 4 mg/kg, about 5 mg/kg, 6 mg/kg, 7 mg/kg, 8 mg/kg, 9 mg/kg, about 10 mg/kg, about 12.5 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg or about 100 mg/kg. It will be appreciated that these dosages are examples only, and an appropriate dose can be determined by one of ordinary skill in the art.

In some examples, viral particles containing the GITR-encoding nucleic acid are administered as part of a preparation having a titer of viral particles of at least 1×10⁵ pfu/ml, at least 1×10⁶ pfu/ml, at least 1×10⁷ pfu/ml, at least 1×10⁸ pfu/ml, at least 1×10⁹ pfu/ml, or at least 1×10¹⁰ pfu/ml, and in some examples not exceeding 2×10¹¹ pfu/ml. The viral particles containing the GITR-encoding nucleic acid molecule can be administered in combination with a pharmaceutically acceptable carrier, for example in a volume up to 10 ml. The pharmaceutically acceptable carrier may be, for example, a liquid carrier such as a saline solution, protamine sulfate or Polybrene.

### Administration of Additional Therapeutic Agents

Combination therapy is encompassed by the present application. The GITR agonist(s) and CpG ODN(s) may be administered to a subject in combination with other biologically active substances or other treatment procedures for the treatment of a tumor. For example, the GITR agonist(s) and CpG ODN(s) can be administered alone or with other modes of treatment. They may be provided before, substantially contemporaneous with, or after other modes of treatment, such as surgery, radiation therapy, chemotherapy and/or biologic therapy. For example, the GITR agonist(s) and CpG ODN(s) can be administered in conjunction with one or more anti-cancer agents, such as a chemotherapeutic agent, growth inhibitory agent, anti-angiogenesis agent, anti-neoplastic composition, biologic agent (e.g., therapeutic mAb), or combinations thereof.

In some examples, the GITR agonist(s) and CpG ODN(s) are used in combination with one or more other agents useful in the treatment of cancer, such as chemotherapeutic agents, biologic agents (e.g., therapeutic monoclonal antibodies), anti-angiogenesis agents, growth inhibitory agents, radiation therapy, surgery, or combinations thereof. Examples of chemotherapeutic agents that can be used include, but are not limited to, alkylating agents such as thiotepa and Cytoxan® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegaI1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, Adriamycin® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., Taxol® paclitaxel (Bristol-Myers Squibb Oncology, Princeton, N.J.), Abraxane® Cremophor-free, albumin-engineered nanoparticle formulation of paclitaxel (American Pharmaceutical Partners, Schaumberg, Ill.), and Taxotere® doxetaxel (Rhône-Poulenc Rorer, Antony, France); chloranbucil; Gemzar® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; Navelbine® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluorometlhylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

Further non-limiting exemplary chemotherapeutic agents include anti-hormonal agents that act to regulate or inhibit hormone action on cancers such as anti-estrogens and selective estrogen receptor modulators (SERMs), including, for example, tamoxifen (including Nolvadex® tamoxifen), raloxifene, droloxifene, 4-hydroxytamoxifen, trioxifene, keoxifene, LY117018, onapristone, and Fareston® toremifene; aromatase inhibitors that inhibit the enzyme aromatase, which regulates estrogen production in the adrenal glands, such as, for example, 4(5)-imidazoles, aminoglutethimide, Megase® megestrol acetate, Aromasin® exemestane, formestanie, fadrozole, Rivisor® vorozole, Femara® letrozole, and Arimidex® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF expression inhibitor (e.g., Angiozyme® ribozyme) and a HER2 expression inhibitor; vaccines such as gene therapy vaccines, for example, Allovectin® vaccine, Leuvectin® vaccine, and Vaxid® vaccine; Proleukin® rIL-2; Lurtotecan® topoisomerase 1 inhibitor; Abarelix® rmRH; and pharmaceutically acceptable salts, acids or derivatives of any of the above.

An “anti-angiogenesis agent” is small molecular weight compound, polynucleotide (including, e.g., an inhibitory RNA (RNAi or siRNA)), polypeptide, isolated protein, recombinant protein, antibody, or conjugate or fusion protein thereof, that inhibits angiogenesis, vasculogenesis, or undesirable vascular permeability, either directly or indirectly. Anti-angiogenesis agents include those agents that bind and block the angiogenic activity of the angiogenic factor or its receptor. In one example, an anti-angiogenesis agent is an antibody or other antagonist to an angiogenic agent, e.g., antibodies to VEGF-A (e.g., bevacizumab (Avastin®)) or to the VEGF-A receptor (e.g., KDR receptor or Flt-1 receptor), anti-PDGFR inhibitors such as Gleevec® (Imatinib Mesylate), small molecules that block VEGF receptor signaling (e.g., PTK787/ZK2284, SU6668, Sutent®/SU11248 (sunitinib malate), AMG706, or those described in, e.g., international patent application WO 2004/113304). Anti-angiogensis agents also include native angiogenesis inhibitors, e.g., angiostatin, endostatin, etc. See, e.g., Klagsbrun and D'Amore (1991) Annu. Rev. Physiol. 53:217-39; Streit and Detmar (2003) Oncogene 22:3172-3179 (e.g., Table 3 listing anti-angiogenic therapy in malignant melanoma); Ferrara & Alitalo (1999) Nature Medicine 5(12): 1359-1364; Tonini et al. (2003) Oncogene 22:6549-6556 (e.g., Table 2 listing known anti-angiogenic factors); and, Sato (2003) Int. J. Clin. Oncol. 8:200-206 (e.g., Table 1 listing anti-angiogenic agents used in clinical trials).

A growth inhibitory agent is to a compound or composition that inhibits growth of a cell (such as a cell expressing VEGF) either in vitro or in vivo. Thus, the growth inhibitory agent may be one that significantly reduces the percentage of cells (such as a cell expressing VEGF) in S phase. Examples of growth inhibitory agents include, but are not limited to, agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (Taxotere®, Rhone-Poulenc Rorer), derived from the European yew, is a semisynthetic analogue of paclitaxel (Taxol®, Bristol-Myers Squibb). Paclitaxel and docetaxel promote the assembly of microtubules from tubulin dimers and stabilize microtubules by preventing depolymerization, which results in the inhibition of mitosis in cells.

Anti-neoplastic compositions include those useful in treating cancer and include at least one active therapeutic agent. Examples of therapeutic agents include, but are not limited to, e.g., chemotherapeutic agents, growth inhibitory agents, cytotoxic agents, agents used in radiation therapy, anti-angiogenesis agents, cancer immunotherapeutic agents, apoptotic agents, anti-tubulin agents, and other biologic agents to treat cancer, such as anti-HER-2 antibodies, anti-CD20 antibodies, an epidermal growth factor receptor (EGFR) antagonist (e.g., a tyrosine kinase inhibitor), HER1/EGFR inhibitor (e.g., erlotinib (Tarceva®), platelet derived growth factor inhibitors (e.g., Gleevec® (Imatinib Mesylate)), a COX-2 inhibitor (e.g., celecoxib), interferons, CTLA4 inhibitors (e.g., anti-CTLA antibody ipilimumab (YERVOY®)), PD-1 inhibitors (e.g., anti-PD1 antibodies, BMS-936558), PDL1 inhibitors (e.g., anti-PDL1 antibodies, MPDL3280A), PDL2 inhibitors (e.g., anti-PDL2 antibodies), TIM3 inhibitors (e.g., anti-TIM3 antibodies), cytokines, antagonists (e.g., neutralizing antibodies) that bind to one or more of the following targets ErbB2, ErbB3, ErbB4, PDGFR-beta, BlyS, APRIL, BCMA, PD-1, PDL1, PDL2, CTLA4, TIM3, or VEGF receptor(s), TRAIL/Apo2, and other bioactive and organic chemical agents, etc. Combinations thereof can also be used.

Other examples of biologic agents to treat cancer that can be used with the GITR agonist(s) and CpG ODN(s) include but are not limited to: cetuximab, brentuximab vedotin, daratumumab, ibritumomab tiuxetan, ipilimumab, nivolumab, ofatumumab, panitumumab, pembrolizumab, rituximab, tositumomab, and trastuzumab.

### Therapeutic Response

Exemplary tumors, such as cancers, that can be treated with the disclosed methods include solid tumors, such as a carcinoma, blastoma, or sarcoma. Specific examples of tumors that can be treated with the disclosed methods include but are not limited to: breast carcinomas (e.g. lobular and duct carcinomas, such as a triple negative breast cancer, and invasive carcinoma), carcinomas of the lung (e.g., non-small cell carcinoma, small-cell lung cancer, large cell carcinoma, squamous carcinoma, and adenocarcinoma), mesothelioma of the lung, colorectal adenocarcinoma (such as colon cancer), stomach carcinoma, prostatic adenocarcinoma, ovarian carcinoma (such as serous cystadenocarcinoma and mucinous cystadenocarcinoma), ovarian germ cell tumors, testicular carcinomas and germ cell tumors, pancreatic adenocarcinoma, biliary adenocarcinoma, hepatocellular carcinoma, cancer of the peritoneum, bladder carcinoma (including, for instance, transitional cell carcinoma, adenocarcinoma, and squamous carcinoma and bladder urothelial carcinoma), thyroid cancer, cholangiocarcinoma, gallbladder carcinoma, renal cell adenocarcinoma, renal clear cell carcinoma, endometrial or uterine carcinomas (including, e.g., adenocarcinomas and mixed Mullerian tumors (carcinosarcomas)), carcinomas of the cervix, endocervix, ectocervix, vulva, and vagina (such as adenocarcinoma and squamous carcinoma of each of same), prostate cancer, hepatoma, cholangiocarcinoma, tumors of the skin (e.g., squamous cell carcinoma, basal cell carcinoma, malignant melanoma, skin appendage tumors, Kaposi sarcoma, cutaneous lymphoma, skin adnexal tumors and various types of sarcomas and Merkel cell carcinoma), esophageal carcinoma, carcinomas of the nasopharynx and oropharynx (including squamous carcinoma and adenocarcinomas of same), salivary gland carcinomas, brain and central nervous system tumors (including, for example, tumors of glial, neuronal, and meningeal origin, pituitary cancer, glioblastomas, and astrocytomas), tumors of peripheral nerve, soft tissue sarcomas and sarcomas of bone and cartilage, head and neck squamous cell carcinoma, and lymphatic tumors (including B-cell and T-cell malignant lymphoma). In one example, the tumor is an adenocarcinoma. In one example, the tumor is a lymphoma.

In some examples, the disclosed methods decrease the size or volume of the injected tumor by at least 30%, at least 40%, at least 50%, at least 60%, at least 70%, at least 80%, at least 90%, at least 95%, at least 98%, at least 99%, or even 100% (e.g., no detectable tumor cells), for example as compared to no administration of the GITR agonist and CpG ODN. In some examples, the disclosed methods decrease the size or volume of the a non-injected metastatic tumor by at least 30%, at least 40%, at least 50%, at least 60%, at least 70%, at least 80%, at least 90%, at least 95%, at least 98%, at least 99%, or even 100% (e.g., no detectable tumor cells) for example as compared to no administration of the GITR agonist and CpG ODN. In some examples, the disclosed methods decrease the number of non-injected metastatic tumors by at least 30%, at least 40%, at least 50%, at least 60%, at least 70%, at least 80%, at least 90%, at least 95%, at least 98%, at least 99%, or even 100% (e.g., no detectable tumor cells), for example as compared to no administration of the GITR agonist and CpG ODN.

In one example, the methods and compositions provided herein, decreases tumor progression, such as the rate of such progression (for example, decreases of at least 5%, at least 10%, at least 20%, or at least 50%, for example relative to no administration of, or prior to administration of the GITR agonist and CpG ODN.

In one example, a therapeutic response is to reduce the number of T regulatory cells in a subject to whom the therapy is administered. For example, a composition can decrease the number of T regulatory cells by at least 10%, at least 20%, at least 30%, at least 40%, at least 50%, at least 60%, at least 70%, at least 80%, at least 90%, at least 95%, at least 98%, or 100%, as compared to the number of T regulatory cells in the absence of the therapeutic composition (or prior to treatment).

In one example, a therapeutic response is to increase the activity of effector T cells in a subject to whom the therapy is administered. For example, a therapeutic composition can increase the activity of effector T cells by at least 10%, at least 20%, at least 30%, at least 40%, at least 50%, at least 60%, at least 70%, at least 80%, at least 90%, at least 100%, at least 2-fold, at least 5-fold, at least 10-fold, or at least 20-fold, as compared to the activity of effector T cells in the absence of the therapeutic composition (or prior to treatment).

In one example, a therapeutic response is to reduce the suppressive activity and lineage stability of T regulatory cells in a subject to whom the therapy is administered. For example, a therapeutic composition can reduce the suppressive activity and lineage stability of T regulatory cells by at least 10%, at least 20%, at least 30%, at least 40%, at least 50%, at least 60%, at least 70%, at least 80%, at least 90%, at least 95%, at least 98%, or 100%, as compared to the suppressive activity and lineage stability of T regulatory cells in the absence of the therapeutic composition (or prior to treatment).

One skilled in the art will appreciate that combinations of these therapeutic responses can be achieved with the disclosed methods and compositions.

Thus, in some examples, the disclosed methods include measuring the size or volume of the injected tumor, the size or volume of a non-injected metastatic tumor, the number of non-injected metastatic tumors, the progression of a tumor, the number of T regulatory cells, the suppressive activity and lineage stability of T regulatory cells, the activity of effector T cells, or combinations thereof, for example over a period of time (such as before and after administration of the therapeutic agent(s))

### Compositions and Kits

Also provided are pharmaceutical compositions and kits for use in the treatment of a tumor. The composition can include one or more GITR agonists (such as a GITR agonist antibody) and one or more CpG ODNs (such as SEQ ID NO: 1, 2, 3 or combinations thereof), for example with at least one pharmaceutical carrier, diluent or excipient. Such compositions can further include other therapeutic agents, such as a chemotherapeutic, biologic, or the like. In some examples, the composition is provided as a lyophilized powder that is reconstituted upon addition of an appropriate liquid, for example, sterile water. In some examples, the composition includes one or more substances that inhibit protein aggregation, such as sucrose and arginine. In some examples, a composition includes heparin and/or a proteoglycan.

Also provided are kits that includes one or more GITR agonists and one or more CpG ODNs (which may be a single composition or separate compositions, for example in separate containers or vials). Such kits can further include one or more chemotherapeutic agents, one or more biologic agents, one or more anti-angiogenesis agents, one or more growth inhibitory agents, one or more anti-neoplastic compositions, or combinations thereof. In some examples, the kits further include a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes. Such additional reagents may be present in separate containers.

Suitable containers include, for example, bottles, vials, syringes, etc. The containers may be formed from a variety of materials such as glass or plastic. In some examples, the container holds a composition which is by itself or combined with another composition effective for treating a tumor and may have a sterile access port (for example the container may be a vial having a stopper pierceable by a hypodermic injection needle).

The kit can include optional components that aid in the administration of the therapeutic agents to patients, such as vials for reconstituting powder forms, syringes for injection, and customized intratumoral delivery systems. The kit may be manufactured as a single use unit dose for one patient, multiple uses for a particular patient (at a constant dose or in which the individual compounds may vary in potency as therapy progresses); or the kit may contain multiple doses suitable for administration to multiple patients (“bulk packaging”). The kit components may be assembled in cartons, blister packs, bottles, tubes, and the like.

Pharmaceutical dosage packs comprising one or more containers, each containing one or more doses of one or more GITR agonists and one or more CPG ODNs, are also provided. In some examples, a unit dosage is provided wherein the unit dosage contains a predetermined amount of a composition(s) comprising the one or more GITR agonists and one or more CPG ODNs, with or without one or more additional agents. In some embodiments, such a unit dosage is supplied in single-use prefilled syringe for injection. In various embodiments, the composition contained in the unit dosage may include saline, sucrose, or the like; a buffer, such as phosphate, or the like; and/or be formulated within a stable and effective pH range.

### Example 1

### Effect of GITR Antibody on Tumor and Metastasis Regression in A20 Model

This example describes methods used to demonstrate that T cell modulation of GITR within the tumor induces a systemic anti-tumor response.

Female BALB/c mice (6-8 weeks old, ˜20 g) were from Jackson Laboratories (Bar Harbor, Me., USA). Mice were shaved prior to tumor cell implantation.

A20 B cell lymphoma cells (ATCC Manassas, Va., USA: Catalog No. TIB-208) were grown in culture at 37° C. in RPMI-1640 (ATCC) with 10% FBS, 1% Penn/Strep, and 0.05 mM 2-mercaptoethanol.

Each mouse was inoculated with two subcutaneous injections of 5×10⁶ A20 cells in 100 μL PBS near the right and left 4th mammary papilla (teat) from the head of the mouse. The length and width of each tumor was measured and the tumor volume calculated according to the formula: tumor volume (mm\hat{ }3)=(length×width\hat{ }2)/2.

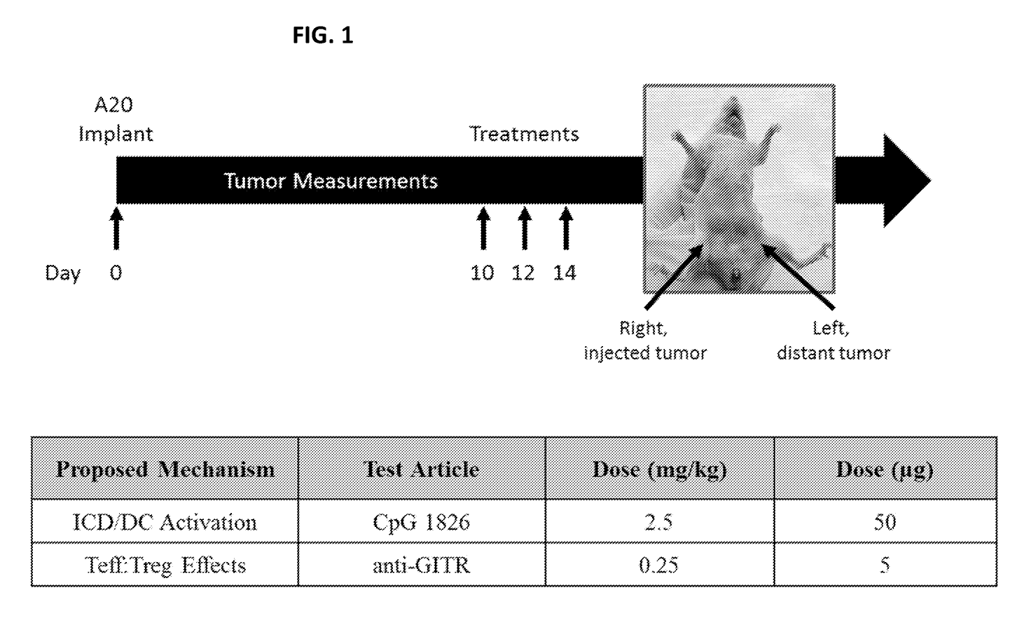

On days 10, 12, and 14, mice were treated with CpG ODN 1826 (CpG, 50 μg, ˜2.5 mg/kg; Adipogen, San Diego, Calif., USA: Catalog No. IAX-200-002) and or anti-GITR (5 μg, ˜0.25 mg/kg; WuXi Biologics, Shanghai, China) as indicated in FIG. 1. Intratumoral injections were combined and given in 25 μL PBS. Intraperitoneal (i.p.) injections of anti-GITR were given in 100 μL PBS.

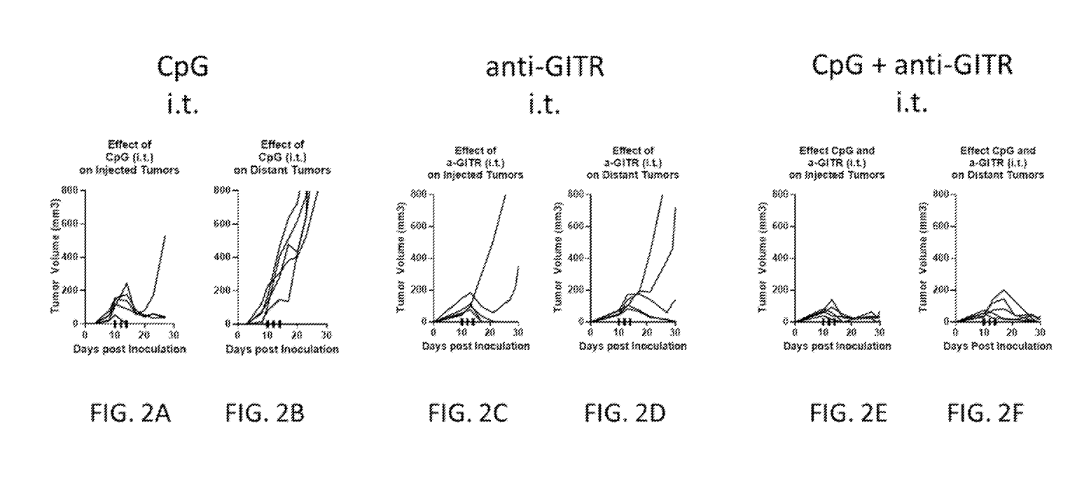

As shown in FIGS. 2E-2F, direct tumor injection of CpG and anti-GITR resulted in tumor regression of both the injected tumor and a distant, non-injected tumor. However, CpG alone caused regression of only the injected tumor (FIG. 2A) but not the distant tumors (FIG. 2B), and anti-GITR alone caused regression of both the injected tumor (FIG. 2C) and distant tumors (FIG. 2D) in some mice.

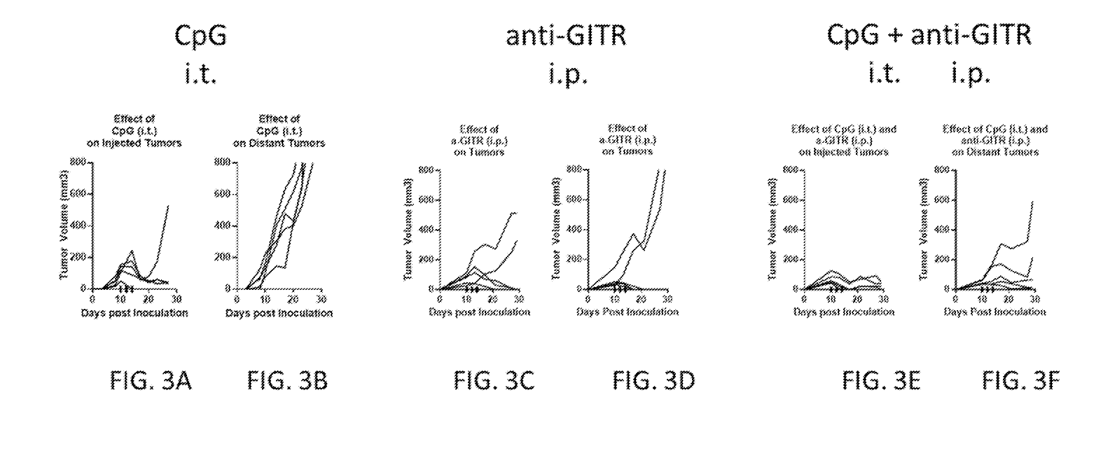

The direct tumor injection of CpG and anti-GITR (FIGS. 2E, 2F) had greater anti-tumor efficacy than CpG and systemic anti-GITR (FIGS. 3E, 3F), which caused tumor regression in some, but not all mice.

### Example 2

### Effect of GITR Antibody on Tumor and Metastasis Regression in CT26 Model

The example above demonstrates not only that intratumoral injection of both CpG and anti-GITR causes tumor regression, but also that intratumoral injection of CpG and systemic administration of anti-GITR (FIGS. 3E, 3F) also cause tumor regression in some mice. Therefore, further experiments will be conducted to show that injection of CpG directly into a tumor increases the response to a systemically administered GITR agonist at a distant, non-injected tumor.

In one example, a CT26 model is used to determine the effect of intratumoral injection of CpG and systemic administration of anti-GITR. Female BALB/c mice (6-8 weeks old) are inoculated in the right and left ventral flank (by the 4th mammary fat pad) with approximately 1×10⁶ CT26 murine colon carcinoma cells (ATCC Manassas, Va., USA; Catalog No. CRL-2638) suspended in 100 μL DPBS. When the tumors reach approximately 100 mm³ (and/or at approximately day 5-7 post-implant), one of the tumors is injected with CpG ODN 1826 (CpG, 10-20 μg, ˜0.5-1.0 mg/kg; Adipogen, San Diego, Calif., USA: Catalog No. IAX-200-002). Agonist anti-GITR is administered intraperitoneally (i.e., systemically) at 0.01-0.1 mg/kg. The tumor volumes of the injected and non-injected tumors are measured, thereby demonstrating that injection of CpG directly into a tumor increases response to systemically administered anti-GITR at a distant, non-injected tumor. The above protocol (including CT26 inoculation and tumor induction, treatment timing and duration, and CpG or anti-GITR dosage) may be adapted for model optimization. Intratumoral injection of both CpG and anti-GITR may also be performed.

An agonist anti-GITR antibody to be used in the above protocol includes any of the anti-GITR antibodies described herein, including a) an antibody comprising a GITR binding domain (GITR-BD) comprising a CDR1 comprising the sequence of SEQ ID NO: 6, a CDR2 comprising the sequence of SEQ ID NO: 7, and a CDR3 comprising the sequence of SEQ ID NO: 8; b) an antibody comprising a GITR-BD comprising the sequence of SEQ ID NO: 5; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 6, a CDR2 comprising the sequence of SEQ ID NO: 7, and a CDR3 comprising the sequence of SEQ ID NO: 8, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:5, (ii) the Linker is a polypeptide, (iii) the Hinge is a polypeptide derived from an immunoglobulin hinge region, and (iv) the Fc is an immunoglobulin Fc polypeptide; or e) a tetravalent molecule comprising two copies of a polypeptide comprising the sequence of SEQ ID NO: 4.

### Example 3

### Effect of GITR Antibody on Tumor and Metastasis Regression in 4T1 Model

In a further example, a 4T1 model is used to determine the effect of intratumoral injection of CpG and systemic administration of anti-GITR. Female BALB/c mice (6-8 weeks old) are inoculated in the right and left ventral flank (by the 4th mammary fat pad) with approximately 5×10⁴ 4 T1 murine mammary carcinoma cells (ATCC Manassas, Va., USA; Catalog No. CRL-2539) suspended in 100 μL DPBS. When the tumors reach approximately 50 mm³ (and/or at approximately day 10-14 post-implant), one of the tumors is injected with CpG ODN 1826 (CpG, 10-20 μg, ˜0.5-1.0 mg/kg; Adipogen, San Diego, Calif., USA: Catalog No. IAX-200-002). Agonist anti-GITR is administered intraperitoneally (i.e., systemically) at 0.01-0.1 mg/kg. The tumor volumes of the injected and non-injected tumors are measured, thereby demonstrating that injection of CpG directly into a tumor increases the response to systemically administered anti-GITR at a distant, non-injected tumor. The above protocol (including 4T1 inoculation and tumor induction, treatment timing and duration, and CpG or anti-GITR dosage) may be adapted for model optimization. Intratumoral injection of both CpG and anti-GITR may also be performed.

An agonist anti-GITR antibody to be used in the above protocol includes any of the anti-GITR antibodies described herein, including a) an antibody comprising a GITR binding domain (GITR-BD) comprising a CDR1 comprising the sequence of SEQ ID NO: 6, a CDR2 comprising the sequence of SEQ ID NO: 7, and a CDR3 comprising the sequence of SEQ ID NO: 8; b) an antibody comprising a GITR-BD comprising the sequence of SEQ ID NO: 5; c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 6, a CDR2 comprising the sequence of SEQ ID NO: 7, and a CDR3 comprising the sequence of SEQ ID NO: 8, (ii) the Linker is a polypeptide, (iii) the Hinge is a polypeptide derived from an immunoglobulin hinge region, and (iv) the Fc is an immunoglobulin Fc polypeptide; d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:5, (ii) the Linker is a polypeptide, (iii) the Hinge is a polypeptide derived from an immunoglobulin hinge region, and (iv) the Fc is an immunoglobulin Fc polypeptide; or e) a tetravalent molecule comprising two copies of a polypeptide comprising the sequence of SEQ ID NO: 4.

Other models may be used to demonstrate that injection of CpG directly into a tumor increases the response to a systemically administered GITR agonist at a distant, non-injected tumor. Such models are responsive to GITR agonists and include models using the B16-F10 murine melanoma cell line (ATCC CRL-6475); MC38 murine colon adenocarcinoma cell line; and EMT6 murine breast mammary carcinoma cell line (ATCC CRL-2755).

In view of the many possible embodiments to which the principles of the disclosure may be applied, it should be recognized that the illustrated embodiments are only examples of the disclosure and should not be taken as limiting the scope of the invention. Rather, the scope of the disclosure is defined by the following claims. We therefore claim as our invention all that comes within the scope and spirit of these claims.

1. A method of treating a tumor in a mammal, comprising:
administering an effective amount of at least one glucocorticoid-induced TNFR-related protein (GITR) agonist; and
administering intratumorally an effective amount of at least one CpG oligodeoxynucleotide (ODN), thereby treating the tumor in the subject.

2. The method of claim 1, wherein the at least one GITR agonist is administered intratumorally.

3. The method of claim 1, wherein the at least one GITR agonist is administered systemically.

4. The method of claim 1, wherein the method further comprises administering to the subject an effective amount of one or more additional therapeutic agents, wherein the one or more additional therapeutic agents comprise one or more chemotherapeutic agents, one or more biologic agents, one or more anti-angiogenesis agents, one or more growth inhibitory agents, one or more anti-neoplastic compositions, surgery, or combinations thereof.

5. The method of claim 1, wherein the mammal is a human.

6. The method of claim 1, wherein the at least one GITR agonist comprises a GITR antibody or GITR antibody fragment.

7. The method of claim 6, wherein the GITR antibody is a chimeric antibody, a humanized antibody, or a human antibody.

8. The method of claim 6, wherein the GITR antibody is a bispecific antibody or a single chain antibody.

9. The method of claim 6, wherein the at least one GITR agonist is a GITR antibody fragment selected from an Fv, a single-chain Fv (scFv), a Fab, a Fab′, and a (Fab′)₂.

10. The method of claim 6, wherein the at least one GITR agonist is a GITR antibody selected from:
a) an antibody comprising a GITR binding domain (GITR-BD) comprising a CDR1 comprising the sequence of SEQ ID NO: 6, a CDR2 comprising the sequence of SEQ ID NO: 7, and a CDR3 comprising the sequence of SEQ ID NO: 8;
b) an antibody comprising a GITR-BD comprising the sequence of SEQ ID NO: 5;
c) a tetravalent molecule comprising two copies of a polypeptide having the structure (GITR-BD)-Linker-(GITR-BD)-Linker-Hinge-Fc, wherein (i) the GITR-BD comprises a CDR1 comprising the sequence of SEQ ID NO: 6, a CDR2 comprising the sequence of SEQ ID NO: 7, and a CDR3 comprising the sequence of SEQ ID NO: 8, (ii) the Linker is a polypeptide, (iii) the Hinge is a polypeptide derived from an immunoglobulin hinge region, and (iv) the Fc is an immunoglobulin Fc polypeptide;
d) a tetravalent molecule comprising two copies of a polypeptide having the structure (GITR-BD)-Linker-(GITR-BD)-Linker-Hinge-Fc, wherein (i) the GITR-BD comprises the amino acid sequence of SEQ ID NO:5, (ii) the Linker is a polypeptide, (iii) the Hinge is a polypeptide derived from an immunoglobulin hinge region, and (iv) the Fc is an immunoglobulin Fc polypeptide; and
e) a tetravalent molecule comprising two copies of a polypeptide comprising the sequence of SEQ ID NO: 4.

11. The method of claim 1, wherein the at least one GITR agonist comprises a GITR peptide or GITR-encoding nucleic acid molecule.

12. The method of claim 1, wherein the at least one GITR agonist comprises a large molecule.

13. The method of claim 1, wherein the tumor is a lymphoma, melanoma, sarcoma or adenocarcinoma.

14. The method of claim 1, wherein the tumor is a cancer of the breast, liver, spleen, kidney, colon, prostate, lung, central nervous system, head and neck, stomach, pancreas, ovary, cervix, testis, bladder or gallbladder.

15. The method of claim 1, wherein the at least one CpG ODN comprises a class B ODN.

16. The method of claim 15, wherein the class B ODN comprises SEQ ID NO: 1 (5′-tcgtcgttttgtcgttttgtcgtt-3′ with bases that are phosphorothioate), SEQ ID NO: 2 (5′-tccatgacgttcctgacgtt-3′ with bases that are phosphorothioate), SEQ ID NO: 3 (5′-TGACTGTGAACGTTCGAGATGA-3), or combinations thereof.

17. The method of claim 1, wherein the method decreases the size or volume of the injected tumor by at least 30%, at least 40%, at least 50%, at least 60%, at least 70%, at least 80%, at least 90%, or at least 95%.

18. The method of claim 1, wherein the method decreases the size or volume of a non-injected metastatic tumor by at least 30%, at least 40%, at least 50%, at least 60%, at least 70%, at least 80%, at least 90%, or at least 95%.

19. The method of claim 1, wherein the method decreases the number of non-injected metastatic tumors by at least 30%, at least 40%, at least 50%, at least 60%, at least 70%, at least 80%, at least 90%, or at least 95%.

20. The method of claim 1, wherein the at least one GITR agonist and the at least one CpG ODN are administered simultaneously or contemporaneously.

21. The method of claim 1, wherein the method comprises at least two separate intratumoral administrations of an effective amount of at least one GITR agonist and at least two separate intratumoral administrations of an effective amount of at least one CpG ODN.

22. The method of claim 1, wherein the effective amount of the at least one GITR agonist is at least 0.25 mg/kg and the effective amount of the at least one CpG ODN is at least 2.5 mg/kg.

23. The method of claim 1, wherein the effective amount of the at least one GITR agonist is from 0.01 to 0.1 mg/kg, and the effective amount of the at least one CpG ODN is from 0.5 to 1.0 mg/kg.

24. A composition comprising:
one or more GITR agonists; and
one or more CpG ODNs.

25. The composition of claim 24, further comprising a pharmaceutically acceptable carrier.

26. A kit comprising the composition of claim 24, and optionally one or more chemotherapeutic agents, one or more biologic agents, one or more anti-angiogenesis agents, one or more growth inhibitory agents, one or more anti-neoplastic compositions, or combinations thereof.
